(19) United States
(12) Patent Application Publication
(10) Pub. No.: US 2017/0037101 A1
(43) Pub. Date: Feb. 9, 2017

(54) Novel Brain Chemokine Samdori and Use Thereof

(71) Applicant: GHBIO Inc., Daejeon (KR)

(72) Inventors: Cheol-Hee Kim, Daejeon (KR); Doo-Sang Park, Daejeon (KR); Jung-Hwa Choi, Daejeon (KR)

(73) Assignee: GHBIO Inc., Daejeon (KR)

(21) Appl. No.: 15/268,945

(22) Filed: Sep. 19, 2016

Related U.S. Application Data
(63) Continuation-in-part of application No. PCT/KR2015/002527, filed on Mar. 16, 2015.

(30) Foreign Application Priority Data
Mar. 18, 2014 (KR) 10-2014-0031448

(51) Int. Cl. C07K 14/52; G01N 33/50; A01K 67/027; A61K 38/19

(52) U.S. Cl. CPC C07K 14/521; A61K 38/195; G01N 33/5023; A01K 67/0276; A61K 48/00

(57) ABSTRACT

The present invention relates to a pharmaceutical composition containing, as an active ingredient, chemokine-like samdori2 (Sam2) for preventing and treating a mental disease associated with a brain nervous system. By constructing a Sam2-specific knockout zebrafish (wherein, the Sam 2 is expressed in a habenular nucleus) and finding that, through novel tank, scototaxis, and social cohesion tests, the zebrafish shows significantly higher fear and anxiety behaviors than the control; inhibitory neuron expression in neurons overexpressing Sam2 gene is decreased; and a patient due to sam2 gene loss shows autism and the hyperanxiety symptom, the Samdori2 can be usefully applied as an active ingredient for a pharmaceutical composition for preventing and treating a mental disease associated with a brain nervous system.

### INTRODUCTION

This patent application is a continuation-in-part of PCT/KR2015/002527, filed Mar. 16, 2015, which claims the benefit of priority from KR 10-2014-0031448, filed Mar. 18, 2014, the contents of each of which are hereby incorporated by reference in their entireties.

### TECHNICAL FIELD

The present invention relates to a novel samdori2 (Sam2) gene, a composition including the gene for diagnosing, preventing or treating a mental disease, and a method for screening an agent for diagnosing, preventing or treating a mental disease using the gene.

### BACKGROUND ART

It has been known that one out of four persons in world population has a mental disease or a neurological disease in one's life. Also, it has been reported that incidence of a mental disease in KOREA is 31.4% (per a lifetime) and 193% (per a year). It has been also reported that 3 mental diseases occur in all age groups and 6 mental diseases occur in the age of between 15 to 44 (WHO, 2001). Depression is a fourth main cause of the whole diseases and has been assumed to become a second cause following ischemic cardiac diseases in 2020.

The technique to treat mental diseases has been greatly improved for decades, but peoples do not escape from suffer of mental diseases. As a representative example, for schizophrenia and depression, which are major mental diseases, at least one third of the patients consistently have symptoms which are not alleviated even by the current therapy. For remaining cases in which symptoms are alleviated, a considerable ratio of patients has decreased quality of life due to residual symptoms and functional disorders, which becomes a social and economical burden factor. In particular, for schizophrenia, the medicinal therapy, which is a current first-line therapy, is effective in positive symptoms, but less effective in negative symptoms or improvement in cognitive function. Therefore, development of a therapeutic agent having improved efficacy is urgently required.

Depression, i.e., depressive disorder, refers to a disease which shows decline in enthusiasm and depressed feeling as main symptoms and causes various cognition, psychological and physical symptoms, thereby causing decline in abilities to function in daily life. Incidence of depressive disorder per life time is 15%, and particularly about 25% in women, and depressive disorder is a serious disease causing changes in emotion, thinking, physical status, and behaviors. Major depression is not caused by one reason. Rather, emotional disorder occurs by interaction of various types of genes, epigenetic influence, embryological causes, and environmental influence which complexly damages emotion.

Further, anxiety refers to a widely very unpleasant and vaguely uneasy feeling and involves physical symptoms such as heart palpitations and sweat and behavior symptoms such as hypersensitiveness and hovering. The number of patients receiving specialized psychotherapy in hospitals due to depression or anxiety has been increased every year. Moreover, sales of antianxiety drugs are increased by about 200% in 2002. Therefore, it is no exaggeration to say that depression or anxiety takes most parts of mental diseases of contemporary peoples, and many people are suffered from depression and anxiety (Robert. F. Scmidt. Human physiology, p366; Min Seonggil. Latest Psychiatry, pp 238-240; S. V., Pharmacol. Ther., 88, pp 213-227, 2000). When being anxious, the entire brain is in an arousal state, so that disorders occur in behavior of peripheral, an autonomic nervous system, sensation, perception, etc. Examples of organs associated with this function include brains such as a cerebrum limbic system (in particular, hippocampus, and cingulate gyrus), cerebral cortex (in particular, frontal lobe and temporal lobe), hypothalamus, ascending reticular system, and pituitary gland and peripheral organs such as thyroid and adrenal cortex. According to the recent brain imaging study, it has been known that anxiety is associated with disorders in the right hemisphere, frontal lobe, temporal lobe, and occipital lobe (Robert. F. Scmidt. Human physiology, p 366; Min Seonggil. Latest Psychiatry, pp 238-240).

After it has been demonstrated that iproniazid, which has been developed as an antituberculous agent, has the antidepressive effect as a monoamine oxidase inhibitor, development of recent antidepressants is invigorated. Chlorpromazine, which is an antipsychotic drug, has been developed as an antihistamine agent in early 1950s, and demonstrated to have an effect in treatment of schizophrenia. Similarly, imipramine has been synthesized as an antihistamine agent by Geigy Drug Co. (Swiss) in 1955. Therefore, the development has been invigorated. Through these findings, tricyclic antidepressants (TCAs) have been developed, and many additional TCAs such as nortriptyline, doxepin, and clomipramine, as well as amitriptyline and desipramine which have modified tricyclic structures are used. Then, second and third generations of antidepressants have been developed. In early 1980s, tetracyclic compounds which have similar structures and efficacy and are referred to as heterocyclics such as maprotiline and amoxapine have been commercialized. In 1980s, prozac, which is a selective serotonin reuptake inhibitor (SSRI), has been developed and used as a good therapeutic agent. However, it has been reported that prozac has side effects as follows: thirst, constipation, dysuria, visual impairment, and impotence which are anticholinergic actions, are shown; blood pressure, pulse, and cardiac conduction are significantly affected due to the action on the cardiovascular system; and orthostatic hypotension occurs due to block of the 1-adrenergic receptor. Also, it has been reported that the sedation effect is exhibited due to the antihistamine effect. Examples of recently used antidepressants include selective serotonin reuptake inhibitors, serotonin norepinephrine reuptake inhibitors, selective norepinephrine reuptake inhibitors, dopamine and norepinephrine reuptake inhibitors (DNRIs), norepinephrine and serotonin receptor antagonists (NaSSAs), serotonin receptor antagonism and serotonin reuptake inhibitors (SARIs), selective serotonin reuptake enhancers (SSREs), etc.

Since the antidepressants recently used in the treatment show low remission rate, sufficient therapeutic effects may not be obtained through recent drugs, so that it has been estimated that further antidepressant market would shrink. To overcome this, development of new concept of a therapeutic agent which overcomes current low remission rate is required. Antidepressants recently used in the treatment have representative side effects such as sexual dysfunction and weight gain, so that it has been expected that development of a new antidepressant which overcomes such side effects opens a new market. One of other key problems of antidepressants recently used in the treatment is that there is lag-time of several weeks or several months until the drugs show efficacy after administration.

Emotional responses such as fear and anxiety serve an important role in decision making and survival. The habenular nucleus is an area of brain which is located at the top of forebrain and maintains high homology in evolution. It has been known that this brain area controls behavior responses to stress, anxiety, and fear, and it has been particularly known that, in the human, functional disorders in the habenular nucleus is associated with depression, post-traumatic stress disorders, and schizophrenia (Hikosaka, Nat. Rev. Neurosci, 11, p 503-513, 2010; Amo, R. et al, J. Neurosci, 30, 1566-1574, 2010). Also, it has been found that, through the experiment of removing a habenular nucleus tissue in a zebrafish and mouse, the habenular nucleus controls fear and anxiety responses. However, understand and study about a molecular mechanism of the habenular nucleus in regulation of emotion is inadequate. Therefore, the present inventors recognize the problems of typical technology in the prevention and treatment of mental diseases such as anxiety and depression, and have tried to develop a new neuromodulator. Consequently, a novel chemokine-like samdori2 (Sam2) protein expressed in a habenular nucleus is identified. To investigate the function of the Sam2, a Sam2-specific knockout zebrafish, which is prepared by using zinc finger nuclease (ZFN) gene scissor technology, is used for novel tank, scototaxis, and social cohesion tests. Therefore, it has been found that a considerably higher anxiety behavior than the control is shown; inhibitory neuron expression in neurons overexpressing Sam2 gene is decreased; and a patient due to Sam2 gene loss exhibits autism and a hyperanxiety symptom. Consequently, it has been found that the samdori2 can be used to develop a pharmaceutical composition for preventing and treating a mental disease, in particular, depression and autism, and therefore the present invention has been completed.

### DISCLOSURE OF THE INVENTION

### Technical Problem

One object of the present invention is to provide a novel samdori2 gene, a composition including the gene for diagnosing, preventing or treating a mental disease, and a method of for screening an agent for diagnosing, preventing, or treating a mental disease using the gene.

### Technical Solution

In order to achieve the objects, the present invention provides a samdori2 (Sam2) gene having any one of base sequences of SEQ ID NOS: 1-3 and 15-17.

The present invention also provides an expression vector including the samdori2 gene having any one of base sequences of SEQ ID NOS: 1-3 and 15-17.

Further, the present invention provides a transformant obtained by transforming a host cell with the expression vector including the samdori2 gene.

The present invention also provides a method for preparing a recombinant transformant expressing samdori2, the method including:

1) preparing an expression vector including the samdori2 gene; and

2) transforming a host cell with the expression vector.

Further, the present invention provides samdori2 protein encoded by the samdori2 gene.

The present invention also provides samdori2 protein having any one of amino acid sequences of SEQ ID NOS: 4-6 and 18-20.

Further, the present invention provides a pharmaceutical composition for preventing and treating a mental disease containing the expression vector including the samdori2 gene or samdori2 protein.

The present invention also provides a health food for preventing and alleviating a mental disease containing the expression vector including the samdori2 gene or samdori2 protein.

Further, the present invention provides a kit for diagnosing a mental disease including an antisense nucleotide for the samdori2 gene, a primer set or probe, or an aptamer or antibody for the samdori2 protein encoded by the samdori2 gene.

The present invention also provides a method for measuring an expression level of the samdori2 gene for monitoring a mental disease or providing information for diagnosis, the method including:

1) preparing cells isolated from a subject-derived habenular nucleus;

2) measuring an expression level of the samdori2 gene having any one of base sequences of SEQ ID NOS: 1-3 and 15-17 in cells of step 1) according to the present invention; and

3) determining that a mental disease occurs or a risk is increased, when the samdori2 gene of step 2) according to the present invention is deleted or decreased compared to the normal control.

Further, the present invention provides a method for screening an agent for preventing or treating a mental disease, the method including:

1) treating cells expressing the samdori2 gene having any one of base sequences of SEQ ID NOS: 1-3 and 15-17, or the samdori2 protein having a base sequences of SEQ ID NOS: 1-3 and 15-17 with a sample;

2) measuring activity or expression of the samdori2 gene or samdori2 protein in step 1) according to the present invention; and

3) screening a subject sample which increases activity or expression of the samdori2 gene or samdori2 protein in step 2) according to the present invention.

The present invention also provides a model animal for a mental disease in which the samdori2 gene having any one of base sequences of SEQ ID NOS: 1-3 and 15-17 is knocked out in a habenular nucleus of an animal other than a human.

Further, the present invention provides a method for preparing a model animal for a mental disease, the method including:

1) microinjecting a vector expressing a gene scissor specific for the samdori2 gene having any one of base sequences of SEQ ID NOS: 1-3 and 15-17 into a fertilized egg of an animal other than a human;

2) raising the animal in step 1) according to the present invention until the animal becomes an adult, and then breeding the animal with a wild type animal; and

3) screening an animal in which the samdori2 gene is knocked out among the bred animals in step 2) according to the present invention.

### Advantageous Effects

The present invention relates to a novel chemokine-like samdori2 (Sam2) gene and use thereof. By constructing a Sam2-specific knockout zebrafish (wherein, the Sam 2 is expressed in a habenular nucleus) and finding that, through novel tank, scototaxis, and social cohesion tests, the zebrafish shows significantly higher fear and anxiety behaviors than the control; inhibitory neuron expression in neurons overexpressing Sam2 gene is decreased; and a patient due to sam2 gene loss shows autism and the hyperanxiety symptom, the Samdori2 can be usefully applied to development of a pharmaceutical composition for preventing and treating a mental disease associated with a habenular nucleus, in particular autism and depression.

### BRIEF DESCRIPTION OF THE DRAWINGS

FIG. 1 are images showing a SAM gene amino acid sequence which is conserved in vertebrates.

FIG. 2 are images showing a SAM gene amino acid sequence which is conserved in vertebrates.

FIG. 3 inclusive of views a, a′, b, b′, c, c′, d, d′, e, e′, f, f′, g, g′, h, h′, i and j show a specific expression pattern of SAM gene family in a central nervous system of a zebrafish.

FIG. 3, views a, a′, b, b′, c, c′, d, d′, e, e′, f, f′, g, g′, h and h′: identification of a samdori gene family expression pattern in a brain of a zebrafish after 3 days of fertilization.

FIG. 3 views i and j: identification of SAM3a and SAM3b gene expression in a spinal cord of a zebrafish after 24 hours of fertilization.

FIG. 4 inclusive of views a, a′, b, b′, c, d, e, e′, f and g show an expression pattern of Sam2 gene-expressing cells using a marker.

FIG. 5 inclusive of views a, a′ b, b′, c, c′, d, d′, e and e′ show SAM gene family expression pattern in mind bomb (mib).

FIG. 6 inclusive of views a, b, c, d, e and f are images of Sam2 expression.

FIG. 6, views a and b are images showing that the Sam2 gene, among SAM genes, is consistently expressed from early development to the adult phase.

FIG. 6, views c, d, e, and f are images obtained by observing development of efferent axons in a Et(−1otpa:mmGFP)hd1 transformed Sam2 knockout zebrafish.

FIG. 7 inclusive of views a, b, b′, c and c′ is an illustration showing Sam2 gene knockout fish preparation using ZFN, i.e. gene scissor technique.

FIG. 7 view a is an illustration showing gain of mutation in two alleles in which bases of 5 Sam2cnu1 and 17 Sam2cun2 are lost.

FIG. 7, views b and b′ is an illustration showing genotype separation of two alleles by using mutation-specific restriction enzyme (Sam2cnu1) and PCR (Sam2cnu1).

FIG. 7 views c and c′ are images showing normal developed embryos of the control and Sam2 mutant after 3 days of fertilization.

FIG. 8 inclusive of views 8a, 8a′, 8b, 8b′, 8c, 8c′, 8d, 8d′, 8e, 8e′, 8f, 8f′, 8g, 8g′ 8h, 8h′, 8i, 8i′, 8j, 8j′, 8k, 8k′, 8l, 8l′, 8m, 8n, 8m′ and 8n′ are images showing an influence of Sam2 gene loss on a particular neuron development in a Sam2 gene knockout zebrafish.

FIG. 8 views a and a′ and b and b′ are images showing comparison of dopamine neuron marker expression patterns of the control and Sam2 gene knockout zebrafish.

FIG. 8 views c, c′, f, f′, l and l′ are images showing comparison of serotonin neuron marker expression patterns of the control and Sam2 gene knockout zebrafish.

FIG. 8 views d and d′ are images showing comparison of oxytocin neuron marker expression patterns of the control and Sam2 gene knockout zebrafish.

FIG. 8 views e and e′ is an image showing comparison of interpenduncular nucleus expression patterns of the control and Sam2 gene knockout zebrafish.

FIG. 8 views g, g′, h, h′, j and j′ are images showing comparison of various neuron marker expression patterns of the control and Sam2 gene knockout zebrafish.

FIG. 8 views m, m′, n and n′ are images obtained by observing nervous circuits of the control and Sam2 gene knockout zebrafish by using a transformant (Tg[hcrt:EGFP]) in which LC branch axons are connected in a hypothalamus.

FIG. 9 views a, b, c, d, e and f are images showing a novel tank test result by using a Sam2 gene knockout zebrafish and control.

FIG. 9 view a is an image showing a moving distance of a single individual of the Sam2 gene knockout zebrafish and control.

FIG. 9 view b is an image showing an average speed of the Sam2 gene knockout zebrafish and control.

FIG. 9 view c is an image showing abnormal behaviors of the Sam2 gene knockout zebrafish and control.

FIG. 9 view d is an image showing freezing bouts of the Sam2 gene knockout zebrafish and control.

FIG. 10 inclusive of views a, b and c are images showing increase in anxiety by using a Sam2 gene knockout zebrafish through an open tank and scototaxis tests.

FIG. 10 view a is an image showing analysis and comparison after placing the control and Sam2 gene knockout zebrafish in an open tank for 30 minutes to measure thigmotaxis, and then measuring time for staying at the corner and center at one minute interval.

FIG. 10 views b and c show the scototaxis test using the sam2 gene knockout zebrafish, and time for staying at white parts by tracing movement of the control and Sam2 gene knockout zebrafish is analyzed and compared.

FIG. 11 views a, a′, b, b′, c and c′ are images obtained by measuring fear and anxiety through observation of a behavior index of a Sam2 gene knockout zebrafish.

FIG. 12 views a, a′, b, b′, c, c′, d and d′ are images obtained by measuring anxiety responses in group behaviors through observation of social cohesion of a Sam2 gene knockout zebrafish.

FIG. 13 views a, b, c, d, e and f are images showing the anxiety response of a Sam2 gene knockout zebrafish by using a molecular marker.

FIG. 14 views a, b, c, d, e and f are images showing an influence of a human Sam2 gene on excitatory and inhibitory synapses through culture of rat hippocampal neurons.

FIG. 15 views a, b and c show images investigating excitatory neuron molecular marker expression through Sam2 gene overexpression.

FIG. 16 is an image investigating autism and a hyperanxiety symptom due to Sam2 gene loss caused by microdeletion in a human.

### MODE FOR CARRYING OUT THE INVENTION

Hereinafter, the present invention will be described in more detail.

The present invention provides a samdori2 (Sam2) gene having any one of base sequences of SEQ ID NOS: 1-3 and 15-17.

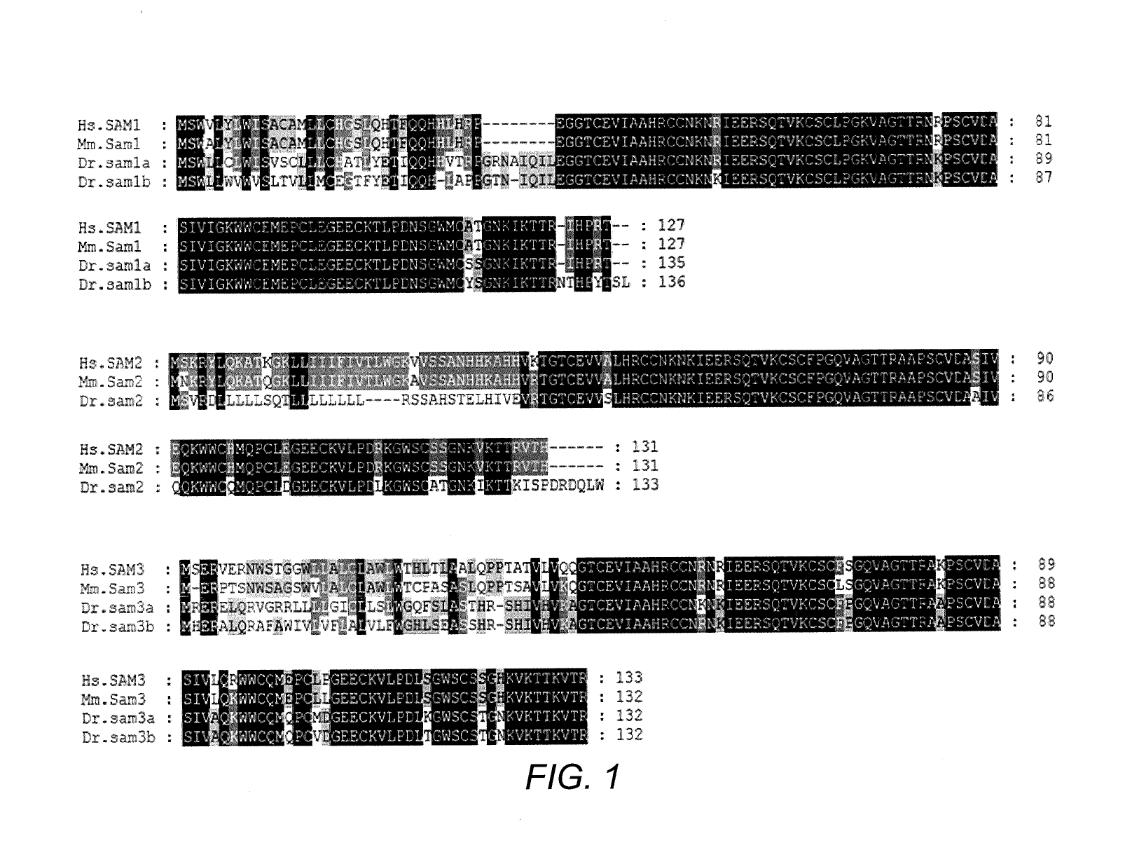

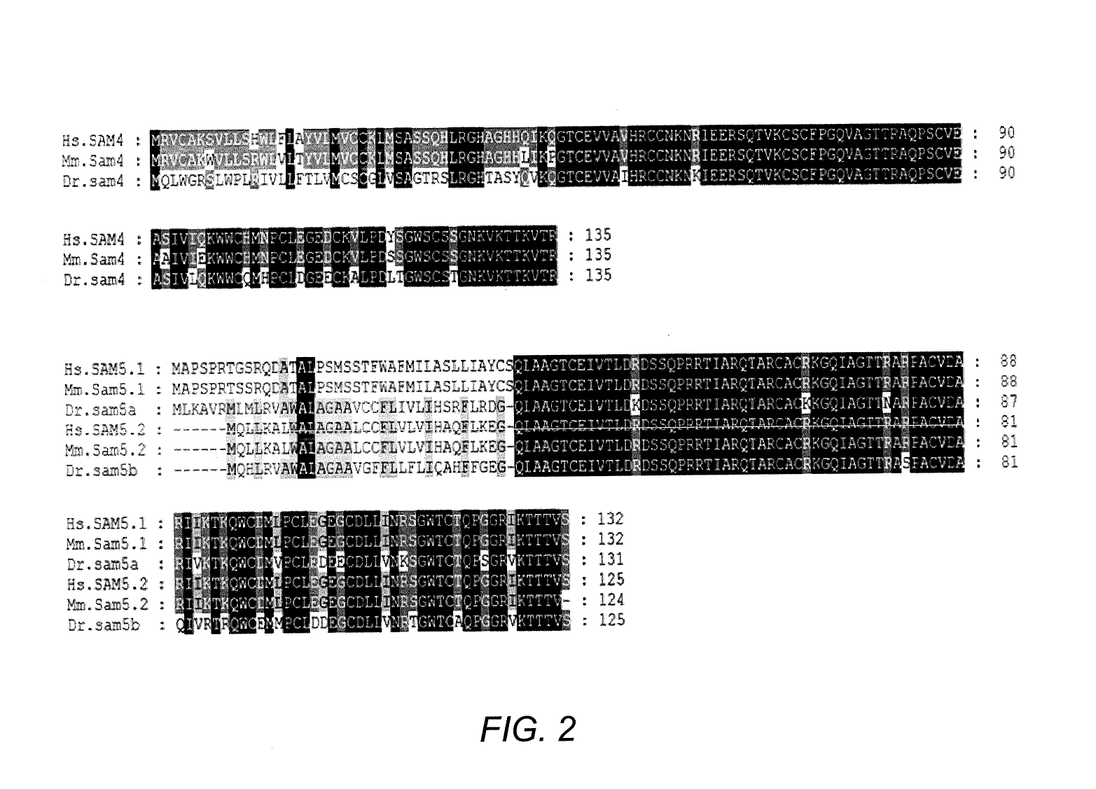

In a particular embodiment of the present invention, to investigate the samdori2 gene expression in a habenular nucleus, the present inventors perform reverse transcription polymerase chain reaction (RT-PCR) for isolating the samdori2 gene from wild type, mind bomb mutant, and Et(−1otpa::mmGFP)hd1 transformed zebrafishes. To investigate evolutional relationship between the gene isolated from the zebrafishes and a genome of a human, similarly on chromosomes is compared by using Synteny database (http://teleost.cs.uoregon.edu/acos/synteny_db/), NCBI (http://www.ncbi.nlm.nih.gov/), Ensembl (http://asia.ensembl.org/), and VEGA (http://vega.sanger.ac.uk/) database. Consequently, 8 genes are found in Sam gene family of the zebrafish and high homology of amino acid sequences between species is also identified (see FIGS. 1 and 2).

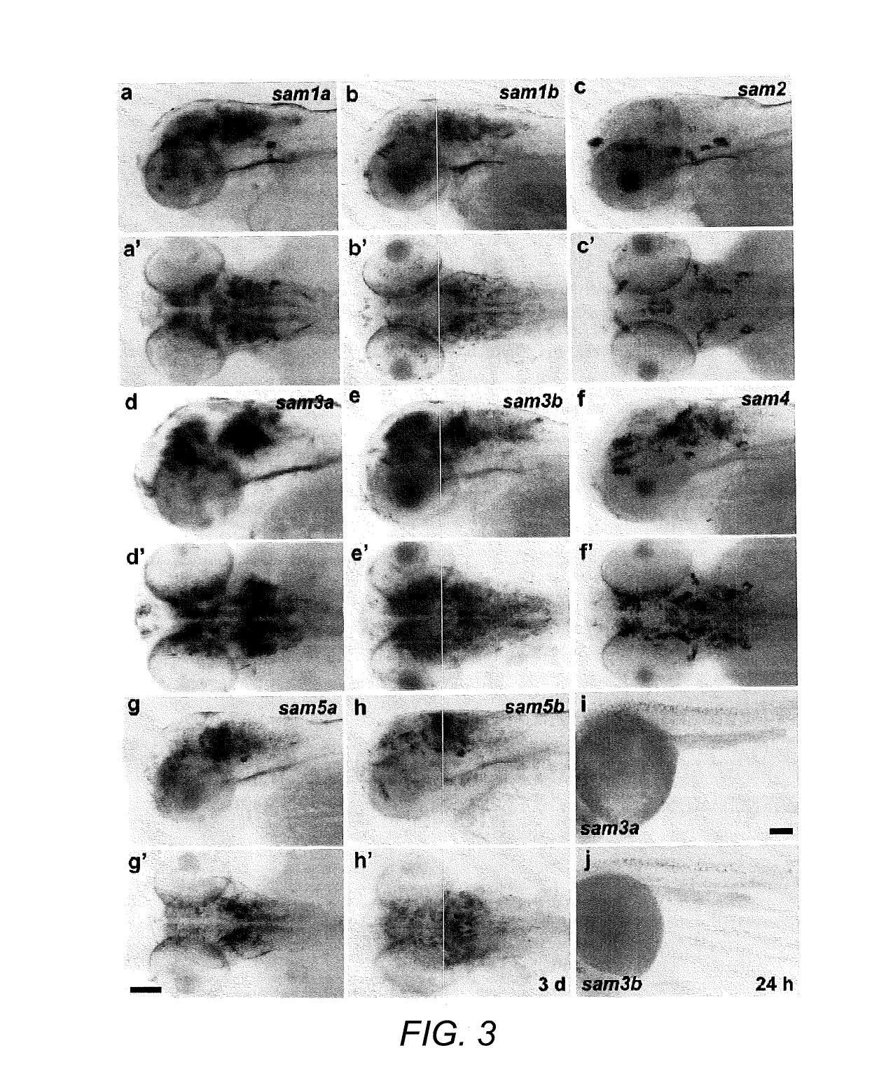

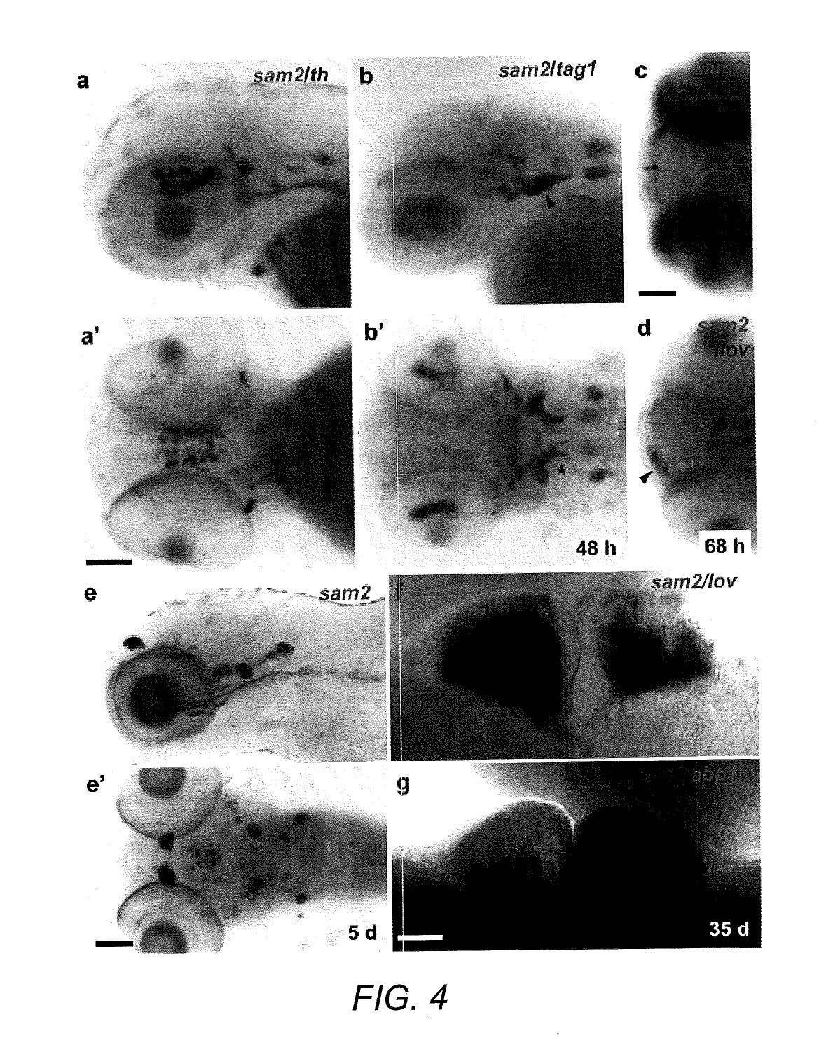

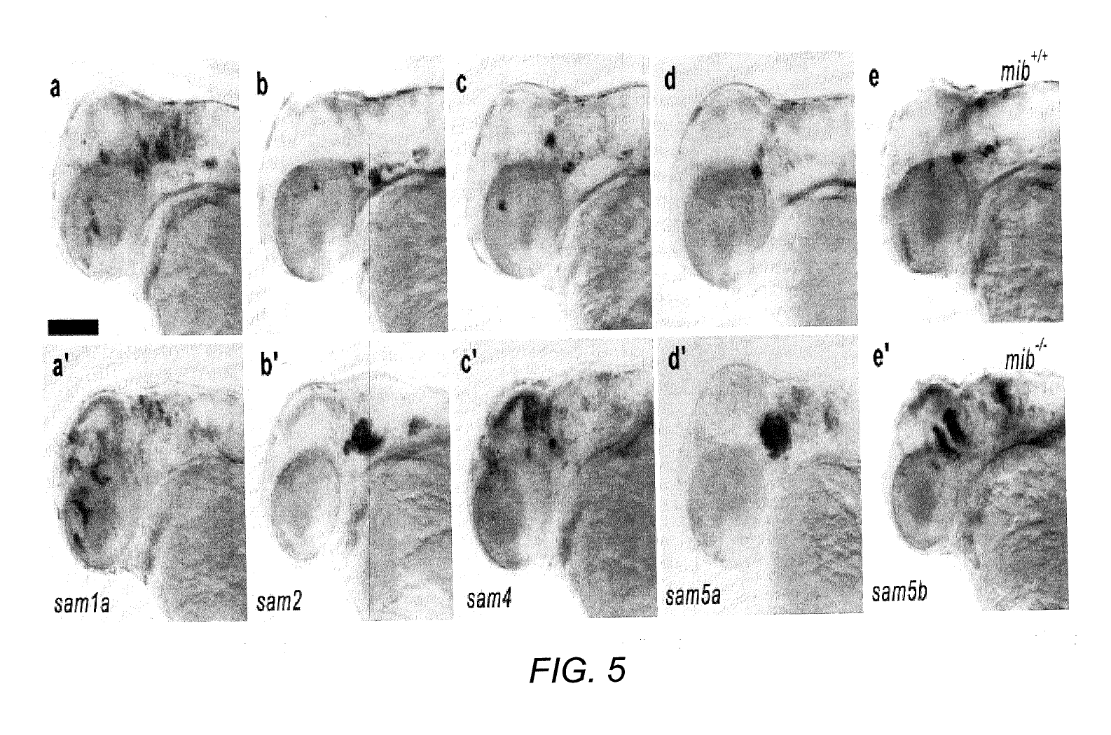

Further, it has been found that Sam gene family has the sequence, CX₇CCX₁₃CXCX₁₄CX₁₁CX₄CX₅CX₁₀C, which is a 10 regular cysteine structure, and this sequence is similar to that of a CC-type chemokine. To investigate a temporal and spatial expression pattern of the isolated Sam2 gene, whole-mount in situ hybridization is performed. As a result, it has been found that all Sam genes are expressed only in a central nervous system (see FIGS. 3 and 4), and cells expressing the Sam genes are neurons through the fact that Sam gene-expressing cells are increased in the mind bomb mutant (mib^{ta52b})¹¹ in which neurons are dramatically increased in the early of development (see FIG. 5).

In addition, it has been found that Sam2, among Sam genes, is expressed in a habenular nucleus from early development to the adult phase (see FIGS. 4 (c-g) and 6 (a-b)).

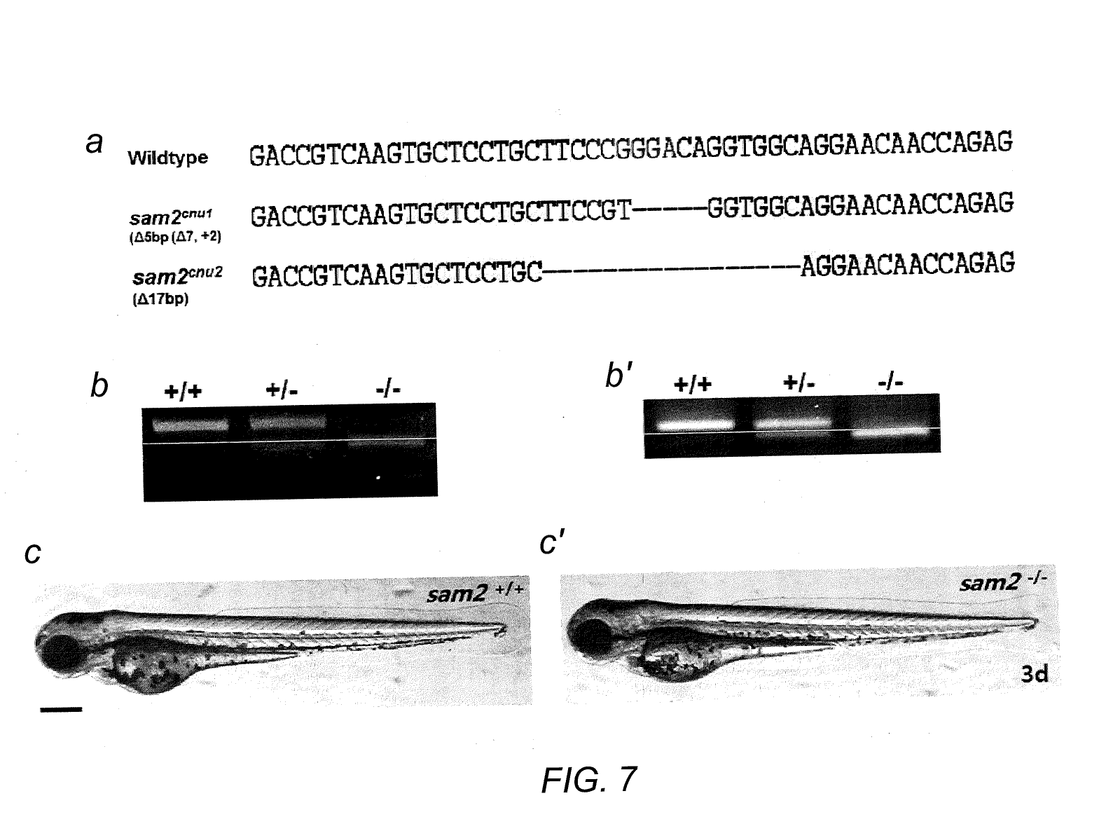

Further, to investigate a function of the Sam2 gene in development of a habenular nucleus and functional regulation, a Sam2 gene knockout (KO) zebrafish is constructed. Consequently, two types of mutants, in which bases of 5 Sam2^{cnu1} and 17 Sam2^{cnu2} are respectively deleted, such that the amino acid of Sam2 is not made, are constructed, and it has been found that the Sam2 knockout zebrafish does not show defects in survival, breeding and morphology (see FIG. 7).

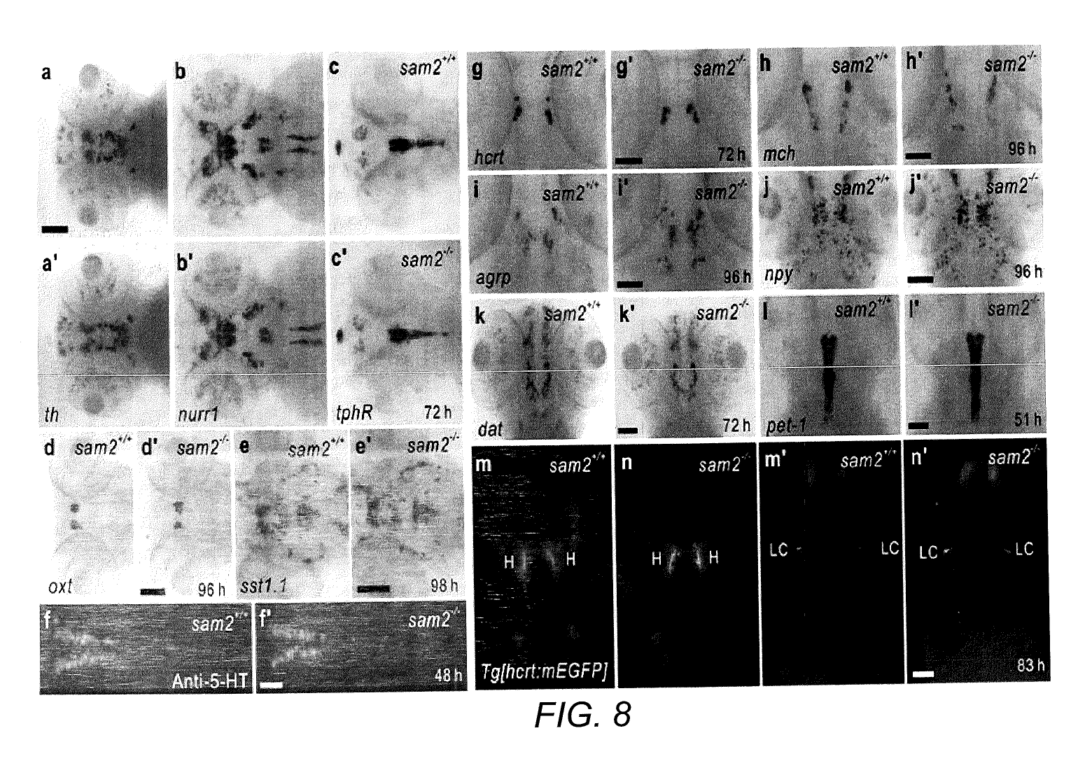

Also, to investigate an influence of Sam2 gene loss in particular neuron development, Sam2 gene loss is compared in a Sam2 gene knockout zebrafish and control by using a dopamine neuron marker, i.e. tyrosine hydroxylase (th), dopamine transporter (dat), nuclear receptor related 1 protein (nurr-1), a serotonin neuron marker (tryptophan hydroxylase Raphe (tphR), 5-hydroxytryptophan (5-HT)), an oxytocin neuron maker (oxytosin(oxt)) and an interpenduncular nucleus maker, i.e., somatostatin1.1 (sst1.1). Consequently, it has been found that Sam2 gene loss of the Sam2 gene knockout zebrafish is not different from that of the control (see FIG. 8).

In addition, to investigate an expression pattern of a gene associated with a neurohormone of a Sam2 gene knockout zebrafish by using various molecular markers, expression patterns of genes associated with a neurohormone are compared in the Sam2 gene knockout zebrafish and control. As a result, it has been found that the expression pattern of the Sam2 gene knockout zebrafish is similar to that of the control (see FIG. 8 (g-l′).

Further, a hypothalamus locus coeruleus projection is observed by using a Tg(hcrt:mEGFP) transformed Sam2 mutant fertilized egg. Consequently, it has been found that the hypothalamus locus coeruleus projection is normal by using the Tg(hcrt:mEGFP) transformed Sam2 mutant fertilized egg (see FIG. 8 (m-n′).

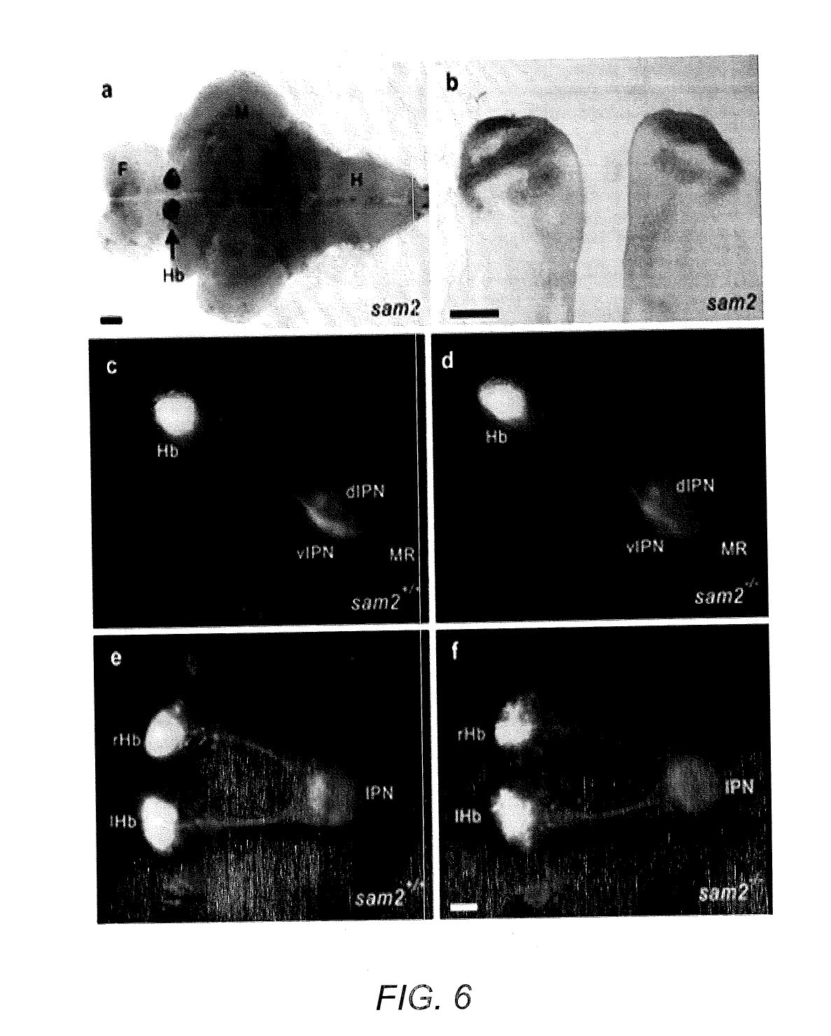

Moreover, efferent axon development is observed in a Et(−1otpa:mmGFP)hd1 transformed Sam2 knockout zebrafish. Consequently, it has been found that axon development extended from a habenular nucleus to an interpenduncular nucleus is normally shown in the Et(−1otpa:mmGFP)hd1 transformed Sam2 knockout zebrafish (see FIG. 6 (c-f)). Therefore, it has been found that the Sam2 gene does not directly affect development and movement of neurons and growth of axons.

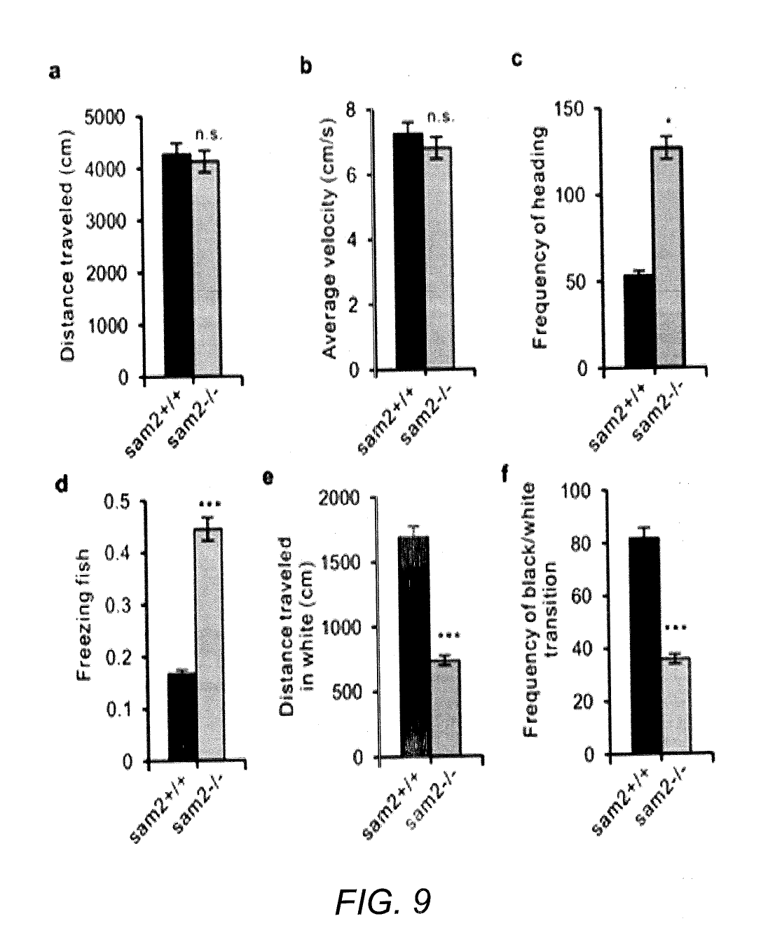

In addition, to investigate an influence of Sam2 gene loss in a zebrafish on fear and anxiety, a Sam2 gene knockout zebrafish is placed on a novel tank and the ability to adapt to a new place is investigated. Consequently, it has been found that, although a locomotor activity such as a moving distance (see FIG. 9a) and average speed (see FIG. 9b) of a single individual of the Sam2 gene knockout zebrafish does not significantly differ from that of the control, anxiety-related behaviors (such as an abnormal behavior of bumping the head into the bottom of the tank (see FIG. 9c) and freezing bouts (see FIG. 9d)) in the Sam2 gene knockout zebrafish is significantly increased when compared to those of the control.

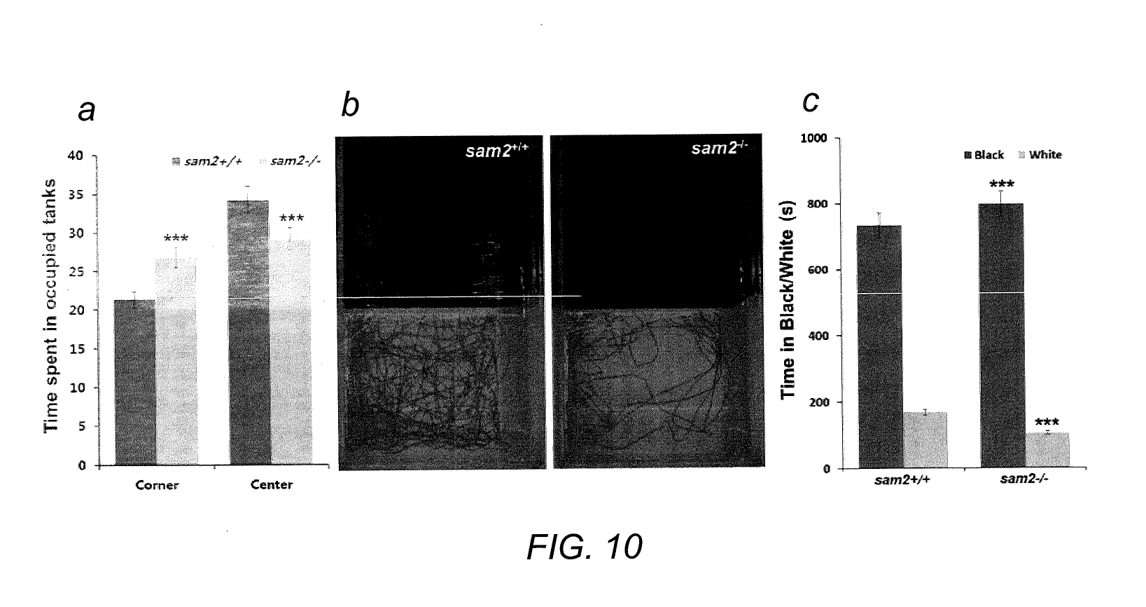

Further, from the thigmotaxis test result, it has been found that the Sam2 gene knockout zebrafish prefers the corner to the center (see FIG. 10). From the results, it has been found that the anxiety symptom of the Sam2 gene knockout zebrafish is observed higher than that of the control. Also, it has been found that, from the scototaxis test result, the anxiety degree of the Sam2 gene knockout zebrafish is higher than that of the control (see FIGS. 9 and 10).

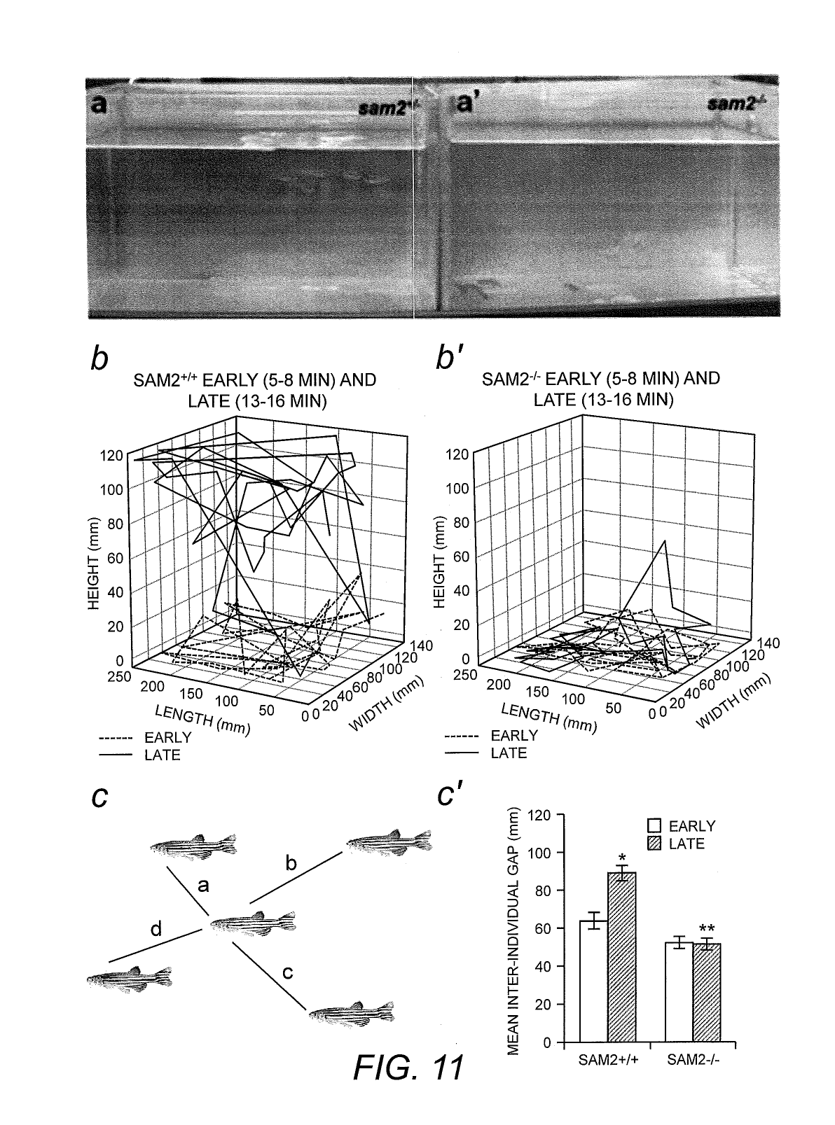

Additionally, to investigate an influence of Sam2 gene loss in a zebrafish on fear and anxiety-associated behaviors, fear and anxiety behaviors of vertebrates are analyzed through a social behavior by using a Sam2 gene knockout zebrafish. Consequently, anxiety behaviors are observed in both the Sam2 gene knockout zebrafish and control during the pre adaptation phase. However, it has been found that, after adaptation, the control adapt to the environment and free swimming is observed, while the Sam2 gene knockout zebrafish does not adapt to the environment and shows continuous anxiety responses (see FIG. 11).

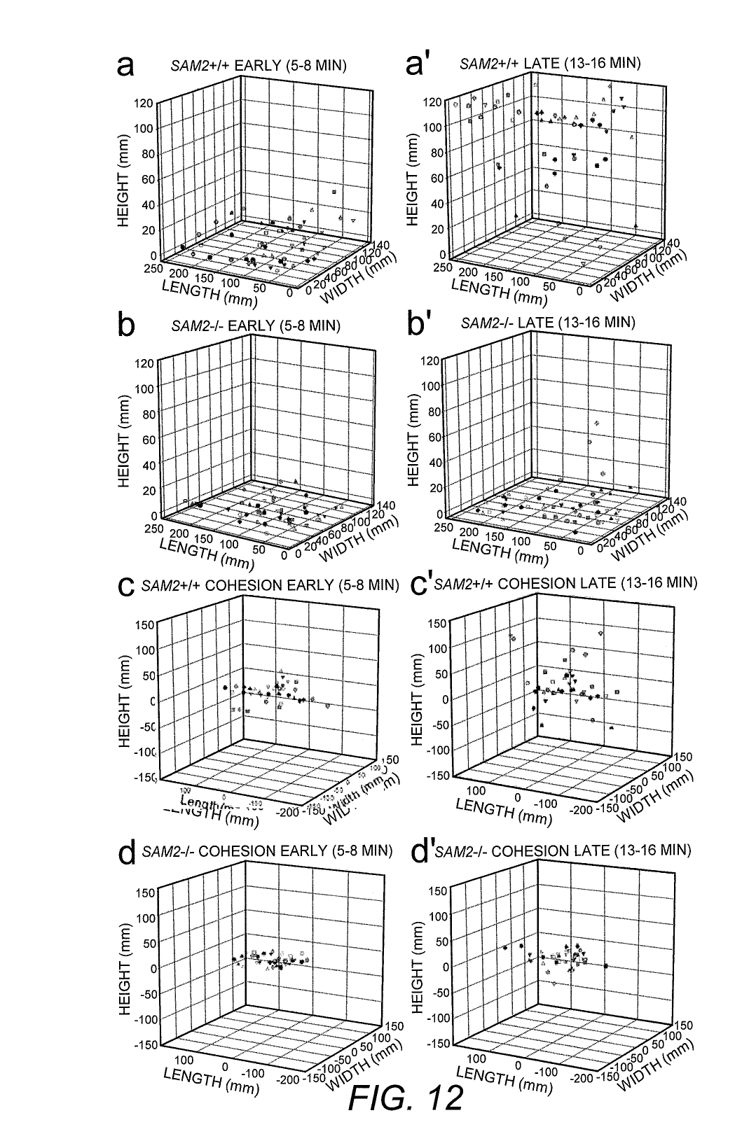

In addition, an anxiety response in group behaviors is measured through observation of social cohesion of a Sam2 gene knockout zebrafish. As a result, it has been found that, for the control, an inter-individual gap between fishes in the group after adaptation is increased than that of before adaptation, while an inter-individual gap between fishes of the Sam2 gene knockout zebrafish does not changed (see FIGS. 11 and 12).

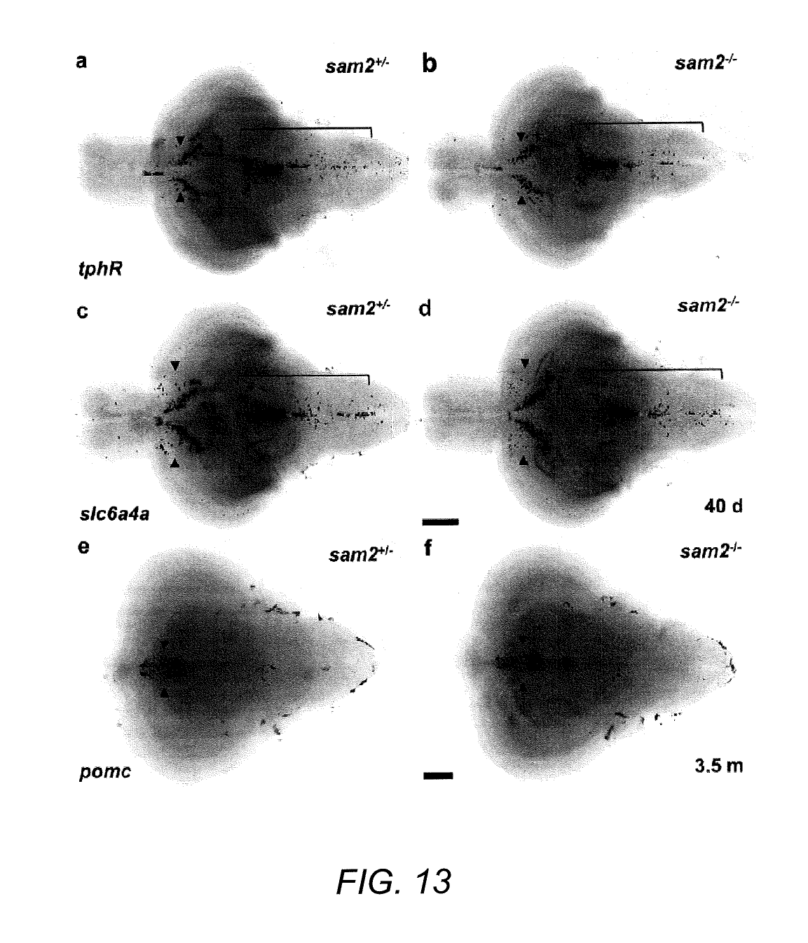

Further, to investigate whether that the anxiety response observed in the Sam2 gene knockout zebrafish is caused by a neurotransmitter, i.e., serotonin system or stress-associated response, analysis is performed by using respective molecular markers. Consequently, it has been found that no difference is observed between anxiety responses of the control and Sam2 gene knockout zebrafish (see FIG. 13). Accordingly, it has been found that the Sam2 gene is involved in fear, anxiety and behavior control in the group. To investigate an influence of the Sam2 gene on behavior control by a habenular nucleus as a neurotransmitter, immunostaining is performed to investigate an influence of a human Sam2 gene on excitatory and inhibitory synapses through culture of rat hippocampal neurons. After overexpression of the Sam2 gene, vesicular GABA transporter (vGAT), gephyrin, and GABA_{A} receptor a2 subunit (GABA_{A}R a2), which are inhibitory synapse markers, are used for investigation. As a result, it has been found that the number and expression level of the inhibitory synapses are significantly reduced relative to the control.

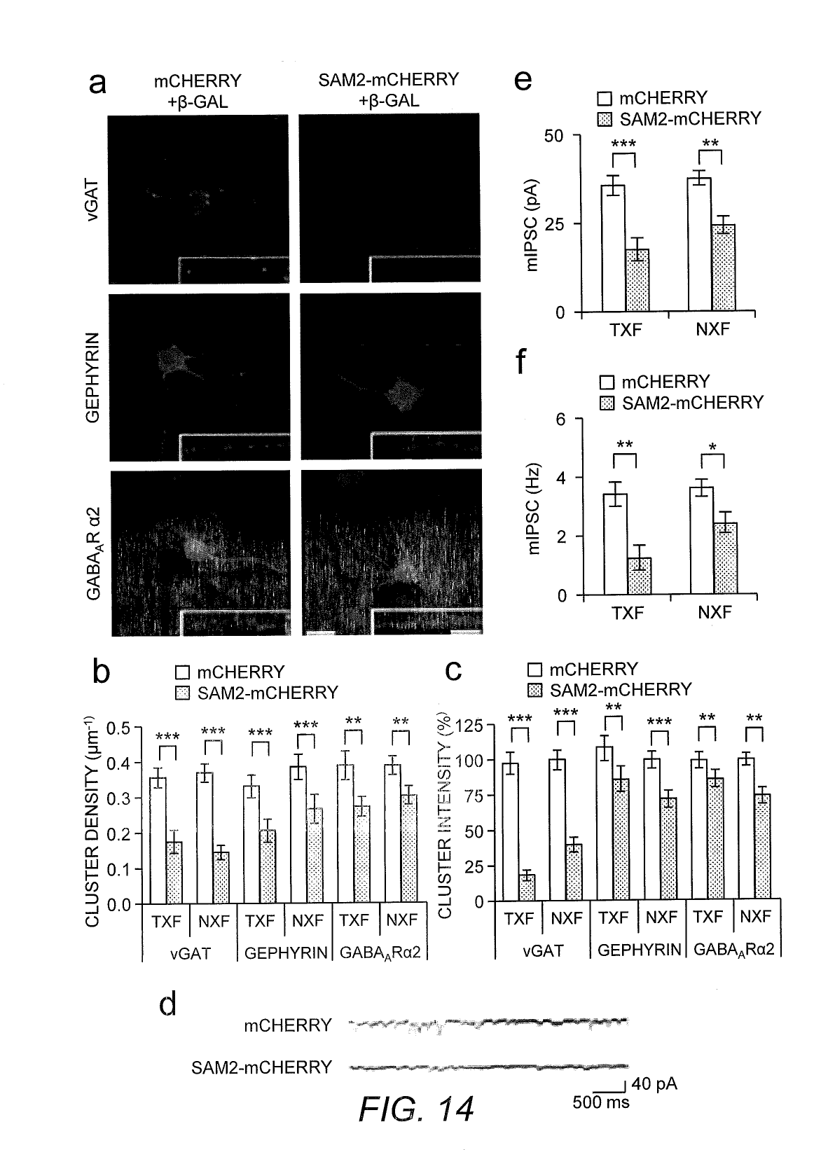

It has been also found that the amplitude and frequency of the miniature inhibitory postsynaptic current (mIPSC) are significantly reduced when the Sam2 gene is overexpressed. In particular, it has been found that the influence of the Sam2 gene on inhibitory synapses affects neighbor neurons as well as the Sam2-treated nerves. Through the results, it has been found that the Sam2 gene is a secretory protein (see FIG. 14).

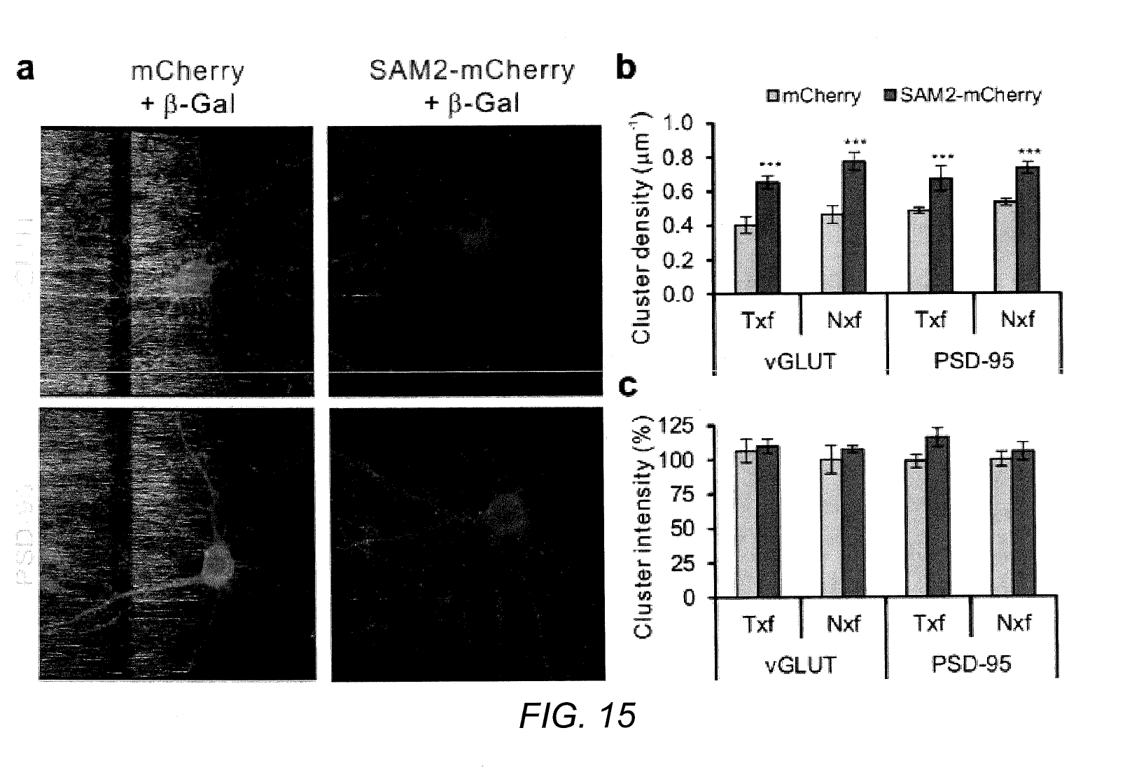

Further, to investigate expression of an excitatory neuromolecular marker due to Sam2 gene overexpression, immunostaining is performed. Consequently, it has been found that the Sam2 gene acts as a neuromodulator and enhances a nerve system activity such as weakening of a presynapse inhibitory function and enhancement of excitatory synapse (see FIG. 15).

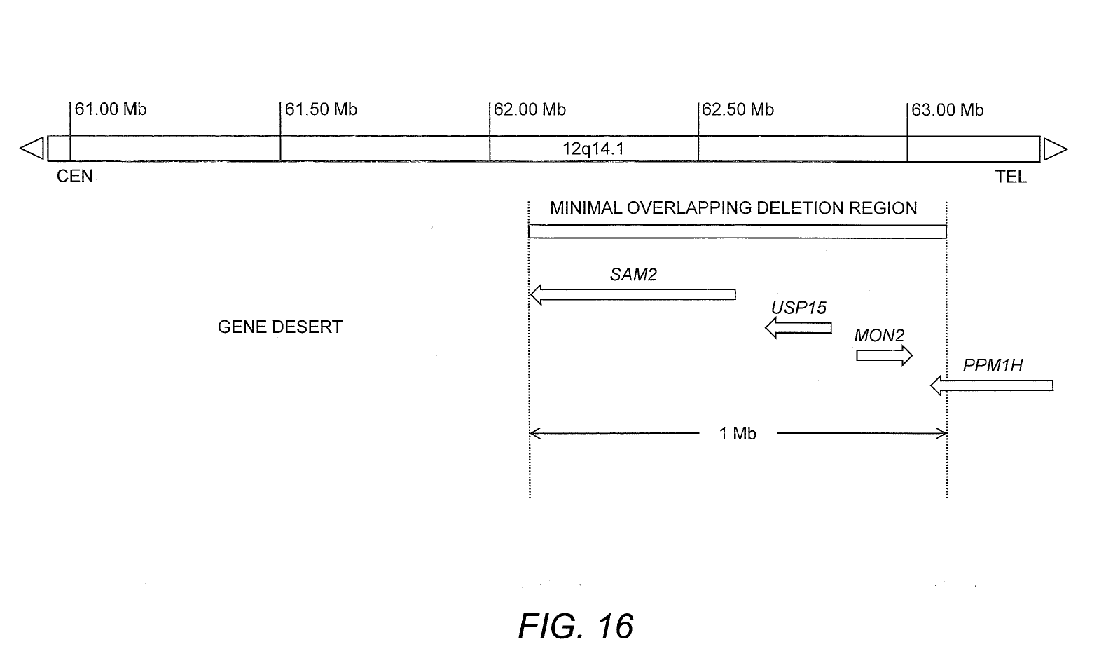

In addition, patients are recruited to investigate autism and a hyperanxiety symptom due to Sam2 gene loss in humans, and autism and the hyperanxiety symptom due to Sam2 gene loss caused by microdeletion are investigated in the recruited patients. Consequently, by identifying autism and the hyperanxiety symptom due to Sam2 gene loss caused by microdeletion in the human, it has been found that the Sam2 gene is a key factor to regulate the anxiety response in the human (see FIG. 16).

Consequently, novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the novel samdori2 gene can be usefully applied to development of a pharmaceutical composition for preventing and treating relating mental diseases, in particular autism and depression.

The present invention also provides an expression vector including the samdori2 gene.

The samdori2 gene preferably includes any one of base sequences of SEQ ID NOS: 1-3 and 15-17, but not limited thereto. Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the expression vector including the novel samdori2 gene can be useful.

Further, the present invention provides a transformant obtained by transforming a host cell with the expression vector.

The present invention also provides a method for preparing a recombinant transformant expressing samdori2, including:

1) preparing the expression vector including the samdori2 gene; and

2) transforming a host cell with the expression vector of step 1) according to the present invention.

A gene of step 1) preferably has any one of base sequences of SEQ ID NOS: 1-3 and 15-17, but not limited thereto.

Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom.

Therefore, the transformant, in which the expression vector including the novel samdori2 gene is transformed, can be useful.

Further, the present invention provides a samdori2 protein encoded by the samdori2 gene.

The present invention also provides a samdori2 protein having any one of amino acid sequences of SEQ ID NOS: 4-6 and 18-20.

Further, the present invention provides a pharmaceutical composition for preventing and treating a mental disease, the composition containing an expression vector including the samdori2 gene or samori2 protein.

The mental disease is preferably anxiety syndrome, depression, autism, manic-depressive illness, schizophrenia, mood disorders, sleep disorders, and attention deficit hyperactivity disorder (ADHD), but not limited thereto. Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the novel samdori2 gene can be usefully applied as a pharmaceutical composition for preventing and treating relating mental diseases, in particular depression and autism.

The composition containing the Sam2 gene of the present invention may contain one or more active ingredients showing the same or similar function in addition to the ingredient described above.

The composition of the present invention may further include a pharmaceutically acceptable additive. As the pharmaceutically acceptable additive, starch, gelatinized starch, microcrystalline cellulose, milk sugar, povidone, colloidal silicon dioxide, calcium hydrogen phosphate, lactose, mannitol, crude maltose, Arabia gum, pregelatinized starch, corn starch, cellulose powder, hydroxypropyl cellulose, opadry, sodium starch glycolate, carnauba wax, synthetic aluminum silicate, stearic acid, magnesium stearate, aluminum stearate, calcium stearate, white sugar, dextrose, sorbitol, talc and so forth may be used. 0.1-90 parts by weight of the pharmaceutically acceptable additive according to the present invention is preferably included with respect to the composition, but not limited thereto.

In other world, the composition of the present invention may be administered in various oral and parenteral formulations for actual clinical administration. For formulation, preparation may be performed by using a normally used diluents or excipients such as a filler, an extender, a binder, a wetting agent, a disintegrating agent, and a surfactant. Examples of solid formulations for oral administration include a tablet, a pill, power, a granule, a capsule, etc. The solid formulations may be prepared by mixing a Common carpesium extract with at least one excipient such as starch, calcium carbonate, sucrose, lactose or gelatin. Further, a lubricant such as magnesium stearate, and talc may be used in addition to the simple excipient. Examples of liquid formulations for oral administration include a suspension, liquid for internal use, an emulsion, and a syrup. In addition to typically used diluents such as water, and liquid paraffin, various excipients such as a wetting agent, a sweetening agent, a flavoring agent, and a preservant may be included. Examples of preparations for parenteral administration may include a sterilized solution, a nonaqueous solvent, a suspension, an emulsion, a lyphophilized preparation, and suppository. As the nonaqueous solvent and suspending solvent, propylene glycol, polyethylene glycol, vegetable oil such as olive oil, injectable ester such as ethyl oleate may be used. As a substrate for the suppository, witepsol, macrogol, tween 61, cacao oil, laurinum, glycerogelati, etc. may be used.

The composition of the present invention may be orally administered or parenterally administered according to the desired method. For parenteral administration, topical skin, intraperitoneal injection, intrarectal injection, subcutaneous injection, intravenous injection, intramuscular injection or intrathoracic injection methods are preferably used. The dose varies depending on weight, age, sex, health status, and diet of patients, administration time, administration methods, excretion rates, and severity of a disease.

The composition according to the present invention is administered in a pharmaceutically effective amount. In the present invention, “a pharmaceutically effective amount” refers to an amount sufficient to treat a disease with a reasonable benefit/risk ratio available to apply a medicinal treatment. The level of effective dose may be determined depending on factors including types of diseases of a patient, severity, activity of a drug, sensitivity to a drug, administration time, administration routes, excretion rates, period of treatment, and a simultaneously used drug, and other factors well known in the medicinal field. The composition of the present invention may be administered as a separate therapeutic agent or administered in combination with other therapeutic agents. Also, the composition of the present invention may be sequentially or simultaneously added with the typical therapeutic agent, and the composition may be for single or multiple administrations. It is important to administer at a minimal dose which may lead a maximum effect without side effects in consideration of all of the factors described above, and the does may be easily determined by a person skilled in the art.

Specifically, an effective amount of the compound according to the present invention may vary depending on age, sex, and weight of a patient. Generally, 0.1 mg to 100 mg, and preferably, 0.5 mg to 10 mg per 1 kg of body weight may be administered daily or every other day, or administered 1 to 3 times per day. However, the dose may be increased or decreased depending on administration routes, severity of obesity, sex, weight, age, and so forth, so that the above dose the does not limit the scope of the present invention in any ways.

The present invention also provides a health food for preventing and alleviating a mental disease, the food containing an expression vector including the samdori2 gene samori2 protein.

The mental disease is preferably anxiety syndrome, depression, autism, manic-depressive illness, schizophrenia, mood disorders, sleep disorders, and attention deficit hyperactivity disorder (ADHD), but not limited thereto. Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the samdori2 gene can be usefully applied as a health food for preventing and alleviating relating mental diseases, in particular depression and autism.

Types of the foods are not specifically limited. Examples of foods into which the above-described material may be added include various foods such as snacks, bread, and noodles, drink such as water, soft drink, and fruit beverage, gum, tea, vitamin complex, seasoning, health functional foods, and include all health functional foods in the typical meaning.

The samdori2 gene of the present invention may be added to foods as it is or used with other foods or food ingredients. Also, the samdori2 gene of the present invention may be appropriately used according to the typical method. The amount of a mixed active ingredient may be suitably determined depending on the purpose of use (for prevention or alleviation). Generally, the compound may be added to the health food in an amount of 0.01 to 15 wt % and preferably 0.1 to 5 wt % with respect to the total weight of the food. For health drink composition, the compound may be added in a ratio of 0.01 to 5.0 g, and preferably 0.01 to 1.0 g based on 100. However, for long term administration for the purpose of health control, the dose may be under the above-described range. Moreover, the active ingredient may be used in an amount above the range described above, because there is no problem in terms of safety.

The health functional drink composition of the present invention does not have specific limitation except the above described compound is included at the specified ratio as an essential ingredient, and includes various flavoring agent or natural carbohydrates may be included as additive ingredients as typical drinks. Examples of natural carbohydrates as described above include typical sugars such as monosaccharides, e.g., glucose, and fructose, disaccharides, e.g., maltose, and sucrose, and polysaccharides such as dextrin, and cyclodextrin; and sugar alcohol such as xylitol, sorbitol, and erythritol. In addition to those described above, natural flavoring agents (e.g., taumakin, and stevia extract) and synthetic flavoring agents (saccharin and aspartame) may be advantageously used. The ratio of the natural carbohydrates is about 0.1 to 2.0 g, and preferably about 0.1 to 1.0 g per 100 g of the composition of the present invention.

The health functional food of the present invention may be easily obtained by adding a process of adding the above described extract or compound of the present invention during the process of preparing a food used as a substrate, or adding a process of adding the above described extract or compound of the present invention after the preparation of a food used as a substrate. Taste and odor adjusting agents may be added.

In addition to those described above, the samdori2 gene of the present invention may contain various nutrients, vitamins, minerals (electrolytes), flavoring agents such as synthetic flavoring agents and natural flavoring agents, coloring agents and bulking agent (chess and chocolate), pectic acid and a salt thereof, alginic acid and a salt thereof, organic acids, protective colloid viscoslty increasing agents, pH adjusting agents, stabilizing agents, preservants, glycerin, alcohol, carbonating agents used in soft drink. In addition to those described above, the samdori2 gene of the present invention may contain fleshes for preparation of natural fruit juice, fruit juice drink, and vegetable drink. These ingredients may be used alone or in combination with other ingredients. Although the ratio of the additive is not important, the samdori2 gene of the present invention is generally selected from the range of about 20 parts by weight per 100 parts by weight.

Further, the present invention provides a kit for diagnosing a mental disease, the kit including an antisense nucleotide for the samdori2 gene, a primer set or probe, or an aptamer or antibody for the samdori2 protein. Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the novel Sam2 gene of the present invention kit may be usefully applied to a kit for diagnosing a mental disease.

The present invention also provides a method for measuring an expression level of the samdori2 gene for providing information for diagnosis or monitoring a mental disease, the method including:

1) preparing cells isolated from a subject-derived habenular nucleus;

2) measuring an expression level of the samdori2 gene in the cells of step 1) according to the present invention; and

3) determining that a mental disease occurs or a risk for a mental disease is increased, when the samdori2 gene of step 2) according to the present invention is deleted or decreased when compared to that of a normal control.

The expression level in step 2) is preferably measured through any one selected from the group consisting of RT-PCR, ELISA, western-blot, and immunohistochemistry, but not limited thereto.

Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the novel Sam2 gene of the present invention may be usefully applied to the method for measuring an expression level of samdori2 gene for providing information for diagnosis or monitoring a mental disease.

Further, the present invention provides a method for screening an agent for preventing or treating a mental disease, the method including:

1) treating cells expressing the samdori2 gene having any one of base sequences of SEQ ID NOS: 1-3, and 15-17, or the samdori2 protein encoded by the samdori2 gene with a subject;

2) measuring activity or expression of the samdori2 gene or samdori2 protein in step 1) according to the present invention; and

3) screening a subject sample which increases activity or expression of the samdori2 gene or samdori2 protein in step 2) according to the present invention.

Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the novel Sam2 gene of the present invention may be usefully applied to the method for screening an agent for preventing or treating a mental disease.

The present invention also provides a model animal for a mental disease in which the samdori2 gene having any one of base sequences of SEQ ID NOS: 1-3 is knocked out in a habenular nucleus of an animal other than the human. The animal is preferably a mouse, a rat, and a zebrafish, but not limited thereto.

The mental disease is preferably anxiety syndrome, depression, autism, manic-depressive illness, schizophrenia, mood disorders, sleep disorders, and attention deficit hyperactivity disorder (ADHD), but not limited thereto. Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control and inhibitory neuron expression is decreased in neurons overexpressing the Sam2 gene. It has been also found that patients caused by Sam2 gene loss show autism and the hyperanxiety symptom. Therefore, the model animal in which the samdori2 gene having any one of base sequences of SEQ ID NOS: 1-3, and 15-17 is knocked out may be usefully applied.

Further, the present invention provides a method for preparing a model animal for a mental disease, including:

1) microinjecting a vector expressing a gene scissor specific for the samdori2 gene having any one of base sequences of SEQ ID NOS: 1-3, and 15-17 into a fertilized egg of an animal other than a human;

2) raising the animal in step 1) according to the present invention until the animal becomes an adult, and then breeding the animal with a wild type animal; and

3) screening an animal in which the samdori2 gene is knocked out among the bred animals in step 2) according to the present invention.

The mental disease is preferably anxiety syndrome, depression, autism, manic-depressive illness, schizophrenia, mood disorders, sleep disorders, and attention deficit hyperactivity disorder (ADHD), but not limited thereto.

Novel tank, scototaxis and social cohesion tests are performed on the novel Sam2 gene of the present invention by using the Sam2 gene-specific knockout zebrafish. It has been found that the zebrafish shows significantly higher fear and anxiety behaviors compared to the control, and inhibitory neuron expression is decreased in neurons overexpressing the Sam2 gene. It has been also found that patients caused by Sam2 gene loss show autism and the hyperanxiety symptom. Therefore, the method for preparing a model animal for a mental disease, in which the samdori2 gene having any one of base sequences of SEQ ID NOS: 1-3, and 15-17 is knocked out, may be usefully applied.

Hereinafter, the present invention will be described in more detail with reference to examples.

However, the following examples are provided to only illustrate the present invention, and the scope of the present invention is not limited to the following examples.

### Example 1

### Raising of Zebrafish and Preparation of Developed Embryo

Wilde type, mind bomb mutant (mibta52b) and Et(−1otpa::mmGFP)hd1 transformed zebrafishes were purchased from an aquarium (Seoul aquarium, Dajeon). The mind bomb (mibta52b) was a mutant obtained through ENU mutagenesis. Mind bomb heterozygote fishes, which is an individual having point mutation in which a RING domain are substituted to M1013R, were inbred and used for the experiment (Itoh, et al., 2003., National Institutes of Health (NIH), USA). The Et(−1otpa::mmGFP)hd1 zebrafish was a novel enhancer trap zebrafish, and provided from Matthias Carl (Germany) and used. The prepared zebrafishes are raised under the following condition: temperature: 28.5° C., light and darkness: light on at 9:00 am to 10:00 pm, and light out other times. Fertilized eggs obtained from the zebrafishes were washed by using egg water (Sea Salts, 280 g), and allowed to develop on petri dishes. Then, the development processes were observed through a dissecting microscope. Developed embryos were selected according to time point and morphological changes, fixed with 4% paraformaldehyde/PBSw.

### Example 2

### Investigation of Sam2 Gene Expression in Habenular Nucleus

<2-1> Isolation of Samdori Gene from Zebrafish

To isolate the samdori gene from wild type, mind bomb mutant and Et(−1otpa::mmGFP)hd1 transformed zebrafishes, which were raised through the method as described in <Example 1>, reverse transcription polymerase chain reaction (RT-PCR) was used.

Specifically, mRNA was isolated by using the wild type raised through the method as described in <Example 1>. Complementary DNA was synthesized from 2 ug of the isolated mRNA by using RT transcript (Enzynomics, Korea). PCR was performed by using the Samdori primer which was synthesized from the complementary DNA and listed in Table 1 below (Table 1). Additionally, the GAPDH primer (SEQ ID NOS: 9 and 10) was used as a control gene (Table 1). After FOR has been completed, the sample was subjected to electrophoresis using 1.0% agarose gel including ethidium bromide, and then UV was irradiated to identify a band. The Sam2 band of 694 bp was cut to isolate DNA from the agarose (Elpis, Korea). The isolated Sam2 DNA was cloned into a T-vector (Promega, USA), and then a plasmid was isolated and purified to analyze the cloned Sam2 DNA gene by using miniprep DNA purification system kit (Intron, Korea). Then, the resultant was analyzed through an automated DNA sequencer.

TABLE 1

| SEQ ID NO. | Sequence |
|---|---|
| sam2, forward | 5′-ACGCCGCTGAATG |
| (SEQ ID NO 7) | AACCGATTACCGG-3′ |
| sam2, reverse | 5′-GAGCGAACGCACT |
| (SEQ ID NO 8) | GCTTTACACAC-3′ |
| beta-actin, forward | 5-GAGGAGCACCCCGT |
| (SEQ ID NO 9) | CCTGCTCAC-3′ |
| beta-actin, reverse | 5-GATGGCTGGAACAG |
| (SEQ ID NO 10) | GGCCTCTGG-3′ |

<2-2> Comparison of Similarity on Chromosome

To investigate evolutional relationship between genomes of the human and zebrafish, the following method was conducted.

Specifically, to investigate evolutional relationship of a genome of a human and a gene isolated from a zebrafish through the method as described in Example <2-1>, amino acid sequences of the Sam2 gene of the human and the Sam2 gene of the zebrafish were searched by using Synteny database (http://teleost.cs.uoregon.edu/acos/synteny_db/), NCBI (http://www.ncbi.nlm.nih.gov/), Ensembl (http://asia.ensembl.org/), and VEGA (http://vega.sanger.ac.uk/) database, and then locations on the chromosomes were compared to investigate evolutional relationship on chromosomes.

Consequently, as shown in FIGS. 1 and 2, 8 genes were found in Sam gene family of the zebrafish, and high homology of amino acid sequences between species was found (FIGS. 1 and 2). It has been also found that the Sam gene family has regular cysteine structure, CX₇CCX₁₃CXCX₁₄CX₁₁CX₄CX₅CX₁₀C sequence, and these sequence has a similar form as CC-type chemokine.

<2-3> Investigation of Time and Spatial Expression Pattern of Sam2 Gene

To investigate a time and spatial expression pattern of the Sam2 gene, whole-mount in situ hybridization was performed.

Specifically, to investigate a time and spatial expression pattern of the Sam2 gene, agrp, cxcr4b, dat, hcrt, lov, mch, npy, otx5, oxt (itnp), pet-1, sst1,th, tphR, and Sam genes were cut with a restriction enzyme, and an anti-sense RNA probe was synthesized by using SP6, T7 or T3 polymerase (Fermenters, USA) and Digoxigenin-labelled UTP (Roche, Germany). Through an optical microscope, after fertilization, an appropriate phase of a fertilized egg at each step was added to 4% paraformaldehyde/PBT and fixed at 4° C. for 12 hours or more. At least 20 hours after fertilization, a chorion of the fertilized egg was removed with tweezers and fixed such that a tail does not bend. For the fertilized egg before 20 hours of fertilization, a chorion was removed after fixing. In addition, from a tail bud phase (10 hours after fertilization), 0.2 mM phenylthiourea/embryonic water (PTU) was added to inhibit skin pigment formation. The fertilized egg, which was fixed after removal of chorion, was washed three times for 5 minutes with 1×PBTw (phosphate buffered saline, 0.1% tween-20), and then replacement was performed about 3 to 4 times by using 100% methanol (MeOH). Then, the resultant was stored at a methanol state at −20° C. The developed embryo stored in methanol was washed in a stepwise manner for 5 minutes by using 75%, 50%, and 25% MeOH/PBSw. Then, washing was performed five times by using PBTw at a 5 minute interval. According to the developmental stage, the developed embryo was treated with PBTw including 10 ug/ml proteinase K. Thereafter, washing was performed 3 to 4 times at a 5 minutes interval by using PBTw. Then, the resultant was fixed with 4% paraformaldehyde/PBT at room temperature for 30 minutes or more. Washing was gently performed five times with PBTw at a 5 minute interval such that the developed embryo does not get damaged. Then, 300 ul HYB (50% formamide, 5×SSC, 0.1% Tween-20) was added and the resultant was stayed for 15 minutes at 68° C. Thereafter, 300 ul of 50% HYB (50% formamide, 5×SSC, 5 mg/ml torula RNA, 50 ug/ml heparin, citric acid, pH6, and 0.1% Tween-20) was added, and the resultant was subjected to prehybridization at 68° C. for 1 to 2 hours or more. Thereafter, about 30 to 100 ng of the probe was added for hybridization at 68° C. for 12 hours. While maintaining a temperature of 68° C., the probe was removed, and washing was sequentially performed for every 15 minutes by using 100%, 75%, 50% and 25% of HYB*/0.2×SSCTw. At the same temperature, replacement with 2×SSCTw was performed and washing was performed for 20 minutes. Then, washing was performed five times for every 30 minutes in 0.2×SSCT to remove a probe which was not hybridized. Thereafter, replacement was sequentially performed by using 75%, 50% and 25% of 0.2×SSCT/PBTw at 5 minute interval, and washing was performed five times for every 5 minutes with PBTw. To investigate color developed and expression regions, 300 ul of 5% sheep serum/PBTw was added, and the resultant was stayed at room temperature for 1 hour, such that a region, where the probe does not bind, was bound to serum to block color development and expression during antibody reaction. Then, 300 ul of anti-DIG-AP Fab (150 u/200 μl; Roche, Germany) diluted to 1/4000 in 5% sheep serum/PBTw was added, and the reaction was performed at room temperature for 4 hours or at 4° C. for 12 to 16 hours. Washing was performed with PBTw six times for 10 minutes, six times for 20 minutes, 8 times for 30 minutes. Thereafter, washing was performed three times for 5 minutes by using a staining buffer (0.1 M Tris-Cl pH 9.5, 0.1 M NaCl, 50 mM MgCl₂, 0.1% Tween-20). A stock of nitro blue tetrazolium/5-bromo 4-chloro 3-indolyl phosphate (NBT/BCIP), which was a chromogenic substrate, was added to a staining buffer in an amount of 4.5 ul/ml (stock: dimethylformamide including 50 mg/ml NBT) or 3.5 ul/ml (stock: dimethylformamide including 50 mg/ml BCIP). Then, reaction was performed at room temperature, while blocking the light, and the stained region was investigated through observation with a microscope. The stained fertilized egg was washed four times for 5 minutes by using a stop solution (1×PBS pH 5.5, 1 mM EDTA, 0.1% Tween-20). Then, washing was performed with 100% methanol for 10 minutes about 4 to 5 times for dehydration, and then the resultant was stored at −20° C. The imaging process was performed by using the MZ-16 microscope (Leica, USA) in a 75% glycerol or benzyl benzoate:benzyl alcohol(2:1) solution.

Consequently, as shown in FIGS. 3 and 4, it has been found that all sam genes are expressed only in a central nervous system (FIGS. 3 and 4).

Also, as shown in FIG. 5, it has been found that cells expressing the sam genes are nerouns through the fact that sam gene-expressing cells are dramatically increased in the mind bomb mutant)(mib^{ta52b}) in which neurons are dramatically increased during the early phase of development (FIG. 5).

Further, as shown in FIGS. 4 (c-g) and 6 (a-b), it has been found that Sam2, among the sam genes, is consistently expressed in a habenular nucleus from the early development to the adult phase (FIGS. 4 (c-g) and 6 (a-b)).

### Example 3

### Investigation of Influence of Sam2 Gene Loss on Particular Neuron Development of Zebrafish

<3-1> Construction of Sam2 Gene Knockout Zebrafish

To investigate the role of Sam2 in development of a habenular nucleus and regulation of a function, a Sam2 gene knockout zebrafish was constructed by using a gene scissor technology, ZFN.

Specifically, to construct zinc-finger nuclease (ZFN) vector, which is a Sam2-specific gene scissor, a third exon of Sam2 was targeted, and zinc fingers respectively corresponding to left ZFN (TGCTCCTGCTTC, SEQ ID NO: 13) and right ZFN (CAGGTGGCAGGA, SEQ ID NO: 14) were used for combination in a modular assembly method to construct the vector. The constructed vector was cut with PvuII restriction enzyme, and ZFN mRNA was synthesized by using T7 RNA polymerase. 2 to 3 ng of the ZFN mRNA constructed through the method was microinjected into a fertilized egg of a zebrafish in one cell stage. The microinjected zebrafish (F0) was raised until the zebrafish became an adult, and then bred with a wild type zebrafish. In the bred zebrafishes, Sam2 gene knockout was screened by using T7 endonuclease 1 method. The genotype of the Sam2 gene knockout zebrafish was screened by using primers listed in Table 2 below.

Consequently, as shown in FIG. 7, two types of mutant, in which bases of 5 Sam2^{cnu1} and 17 Sam2^{cnu2} were respectively damaged such that the amino acid of Sam2 was not made, were obtained, and it has been found that the Sam2 knockout zebrafish does not have defect in survival, breeding and morphology (FIG. 7).

TABLE 2

| SEQ ID NO. | Sequence |
|---|---|
| Sam2^{cnu1} forward | 5′-TCTACTGAGGAGTGGTGTGA-3′ |
| (SEQ ID NO: 11) | |
| Sam2^{cnu1} reverse | 5′-GGTCAGTTTCAGAGAGCTGG-3′ |
| (SEQ ID NO: 12) | |
| left ZFN | TGCTCCTGCTTC |
| (SEQ ID NO: 13) | |
| right ZFN | CAGGTGGCAGGA |
| (SEQ ID NO: 14) | |

<3-2> Investigation of Influence of Sam2 Gene Loss on Particular Neuron Development

To investigate influence of sam2 gene loss on particular neuron development by using the Sam2 gene knockout zebrafish prepared through the method as described in Example <3-1>, the following method was conducted.

Specifically, an influence of sam2 gene loss on particular neuron development was investigated by using a dopamine neuron marker, i.e. tyrosine hydroxylase (th), dopamine transporter (dat), and nuclear receptor related 1 protein (nurr-1), a serotonin neuron marker (tryptophan hydroxylase Raphe (tphR), 5-hydroxytryptophan (5-HT)), an oxytocin neuron marker (oxytosin (oxt)) and an interpenduncular nucleus marker, i.e., somatostatin1.1 (sst1.1). th and dat DNA, which were expressed in dopamine neuron, were cut with a restriction enzyme. Then, in vitro transcription was performed by using RNA polymerase to prepare an mRNA probe. The prepared mRNA probe was used to perform whole-mount in situ hybridization.

Consequently, as shown in FIG. 8, it has been found that, when comparing Sam2 gene loss in the Sam2 gene knockout zebrafish and control, Sam2 gene loss of the Sam2 gene knockout zebrafish is not different from that of the control (FIG. 8).

<3-2> Investigation of Expression Pattern of Gene Associated with Neurohormone of Sam2 Gene Knockout Zebrafish

To investigate an expression pattern of a gene associated with neurohormone of a Sam2 gene knockout zebrafish by using the Sam2 gene knockout zebrafish prepared through the method as described in Example <3-1>, the following method was conducted.

Specifically, DNA of hypocretin/orexin (hcrt), melanin concentrating hormome (mch), agouti related protein homolog (agrp), neuropeptide Y (npy), and fev (ETS oncogene family; pet-1) were cut with a restriction enzyme. Then, in vitro transcription was performed by using RNA polymerase to prepare an mRNA probe. The constructed mRNA probe was used to perform whole-mount in situ hybridization.

Consequently, as shown in FIG. 8 (g-l′), it has been found that, when expression patterns of the gene associated with neurohormone of the Sam2 gene knockout zebrafish and control, the expression pattern of the gene associated with neurohormone of the Sam2 gene knockout zebrafish is similar to that of the control (FIG. 8 (g-l′)).

<3-3> Observation of Hypothalamus Locus Coeruleus Projection by Using Tg(hcrt:mEGFP) Transformed Sam2 Mutant Fertilized Egg

To observe hypothalamus locus coeruleus projection by using a Tg(hcrt:mEGFP) transformed Sam2 mutant fertilized egg, the following method was conducted.

Specifically, an F1 adult zebrafish of Tg(hcrt:mEGFP), which was a transformant expressing a fluorescent protein in a hypothalamus locus coeruleus projection, was bred with a homozygote adult zebrafish having sam2 gene loss. Then, from developed embryos of 48 hours obtained through breeding, an individual expressing fluorescence was selected by using a fluorescent microscope, and raised until the embryo became an adult. Through inbreeding of heterozygote zebrafishes having Sam2 gene loss and expressing fluorescence, a homozygote developed embryo having Sam2 gene loss and expressing fluorescence was obtained. Then, 83 hours after fertilization of homozygote developed embryo, a hypothalamus locus coeruleus projection was observed through a fluorescence microscope.

Consequently, it has been found that the hypothalamus locus coeruleus projection is normal by using Tg(hcrt:mEGFP) transformed Sam2 mutant fertilized egg (FIG. 8 (m-n′)).

<3-4> Observation of Development of Efferent Axon of Et(−1otpa:mmGFP)Hd1 Transformed Sam2 Knockout Zebrafish

To observe development of an efferent axon of Et(−1otpa:mmGFP)hd1 transformed Sam2 knockout zebrafish, the following method was conducted.

Specifically, an adult zebrafish of Et(−1otpa:mmGFP)hd1, which was a transformant expressing a fluorescent protein in a nerve connected to a habenular nucleus, was bred with a homozygote adult zebrafish having sam2 gene loss. Then, from developed embryos of 48 hours obtained through the breeding, an individual expressing fluorescence was selected by using a fluorescent microscope, and raised until the embryo became an adult. Through inbreeding of heterozygote zebrafishes having Sam2 gene loss and expressing fluorescence, a homozygote developed embryo having Sam2 gene loss and expressing fluorescence was obtained. The homozygote developed embryo having Sam2 gene loss obtained through the method was fertilized, and, after 4.5 days of fertilization, a habenular nucleus nerve was observed through a fluorescence microscope.

Consequently, as shown in FIG. 6 (c-f), it has been found that development of an efferent axon extended from a habenular nucleus toward an interpenduncular nucleus was shown in normal in the Et(−1otpa:mmGFP)hd1 transformed Sam2 knockout zebrafish (FIG. 6 (c-f)).

Therefore, it has been found that the Sam2 gene does not directly affect on development and movement of neurons and growth of axons.

### Example 4

### Investigation of Influence of Sam2 Gene Loss on Fear and Anxiety of Zebrafish

To investigate an influence of Sam2 gene loss on fear and anxiety of a zebrafish, open tank, novel tank and dark/light preference (scototaxis) tests were performed on the Sam2 gene knockout zebrafish prepared through the method as described in Example <3-1>.

Specifically, to investigate a fear response, a male Sam2 gene knockout zebrafish prepared through the method as described in Example <3-1> was placed in a novel tank (15 cm>15 cm>25 cm; height>length>width) for 10 minutes, and behaviors were observed through video recording. To perform fear response analysis through the scototaxis test, a zebrafish was placed in a box having white and black regions and allowed to adapt for 5 minutes. Then, behaviors were analyzed through video recording for 15 minutes. To compare the fear responses of the zebrafishes, a heterozygote adult zebrafish without Sam2 gene loss among descendants obtained by inbreeding of heterozygote adult zebrafishes having Sam2 gene loss was used as a control. For the behavior analysis, EthoVision XT7 was used, and statistical analysis was performed through Student's t-test.

Consequently, as shown in FIG. 9, it has been found that, as a result of the novel tank test to investigate an ability to adapt a novel place, a moving distance (FIG. 9a), an average speed (FIG. 9b) and a locomotor activity of a single individual of the Sam2 gene knockout zebrafish are not significantly different from those of the control, however, anxiety responses of the Sam2 gene knockout zebrafish such as an abnormal behavior of bumping a head into the bottom of the tank (FIG. 9c) and freezing bouts (FIG. 9d) are significantly increased.

Further, as shown in FIGS. 9 and 10, it has been found that, trough the thigmotaxis test result, the Sam2 gene knockout zebrafish prefers the corner to the center (FIG. 10). Through the result, it has been found that the anxiety symptom of the Sam2 gene knockout zebrafish is observed higher. Moreover, it has been found that the anxiety degree of the Sam2 gene knockout zebrafish is higher than that of the control through the scototaxis test (FIGS. 9 and 10).

### Example 5

### Investigation of Sam2 Gene Loss on Fear and Anxiety of Zebrafish

<5-1> Measurement of Fear and Anxiety Through Observation of Behavior Index of Sam2 Gene Knockout Zebrafish

To analyze fear and anxiety responses of vertebrates through social behaviors by using the Sam2 gene knockout zebrafish prepared through the method as described in Example <3-1>, the following method was conducted. Specifically, it has been reported that formation of a group called shoaling, which is an innate response of fishes to cope with predators, is used as a behavior index to measure fear and anxiety (Gerlai. R, Behav Brain Res, 2010, 207, 223-231). Therefore, through the social behavior of the Sam2 gene knockout zebrafish in a group, fear and anxiety responses of vertebrates were analyzed. Through the method as described in <Example 4>, 5 Sam2 gene knockout zebrafishes and 5 controls were placed in a novel tank, and flow and locations of the zebrafishes in the group were recorded through a video for 30 minutes and analyzed (FIGS. 11 and 12). Behaviors for 5 to 8 minutes before habitation of the zebrafishes and behaviors for 13 to 16 minutes after habitation were analyzed at a 20 second interval, and an individual gap between fishes was calculated by using the following [Formula 1]. Moreover, statistical analysis was performed by using a SigmaPlot software.

Consequently, as shown in FIG. 11, it has been found that, both the Sam2 gene knockout zebrafish and control show the anxiety responses before adaptation, but after adaptation, the control adapt to the environment and free swimming was observed, while the Sam2 gene knockout zebrafish does not adapt to the environment and shows the continuous anxiety response (FIG. 11).

Distance=\sqrt{(x_{α}−x₀)²+(y_{α}−y₀)²+(z_{α}−z₀)²} [Formula 1]

Wherein, x₀, y₀ and z₀ indicate a fish located at the center in the fish group; and

x_{α}, y_{α} and z_{α} indicate shoaling fishes.

<5-2> Measurement of Anxiety Response in Group Behavior Through Observation of Social Cohesion of Sam2 Gene Knockout Zebrafish

To measure the anxiety response in group behaviors through observation of social cohesion of the Sam2 gene knockout zebrafish prepared through the method as described in Example <3-1>, the following method was conducted.

Specifically, social cohesion which measures an inter-individual gap between fishes in a group is an index for the anxiety response in group behaviors. The anxiety response of the Sam2 gene knockout zebrafish was analyzed through the method as described in Example <5-1>, and an individual gap between fishes was calculated by using the above [Formula 1]. Moreover, statistical analysis was performed by using the SigmaPlot software.

Consequently, as shown in FIGS. 11 and 12, it has been found that, for the control, the inter-individual gap between fishes in the group after adaptation is increased than that of before adaptation, while the inter-individual gap between Sam2 gene knockout zebrafishes does not changed (FIGS. 11 and 12).

<5-3> Investigation of Anxiety Response of Sam2 Gene Knockout Zebrafish by Using Molecular Marker

To investigate whether the anxiety response observed in the Sam2 gene knockout zebrafish prepared through the method as described in Example <3-1> is caused by a neurotransmitter, serotonin system, or stress-related response, corresponding molecular markers were used.

Specifically, to isolate brains of one month and 3 months of heterozygote and homozygote having Sam2 gene loss, each fish was placed in a 15 ml tube, and fixed with 4% paraformaldehyde/PBTw. After 12 hours, the brain of the fish was isolated and dehydrated with methanol. Then, whole-mount in situ hybridization was performed. DNAs of tphR, slc6a4a and pomca were cut, and an mRNA probe was constructed through in vitro transcription by using RNA polymerase. Whole-mount in situ hybridization was performed by using the constructed mRNA probe.

Consequently, as shown in FIG. 13, no difference was observed in the anxiety responses of the control and Sam2 gene knockout zebrafish (FIG. 13).

Therefore, it has been found that the Sam2 gene is associated with fear, anxiety and behavior control in the group through the result of Example <5-3>.

### Example 6

### Investigation of Influence of Sam2 Gene on Behavior Control by Habenular Nucleus as Neurotransmitter

<6-1> Investigation of Influence of Human Sam2 Gene on Excitatory and Inhibitory Synapses Through Rat Hippocampal Neuron Culture

To investigate whether the Sam2 gene is associated with behavior control by a habenular nucleus as a neurotransmitter, immunostaining was performed.

Specifically, hippocampal neurons on Day 18 of BALB/c mouse embryo development were cultured in a B27 cell culture medium (Animal Experiment committee of Medical College of Wisconsin, IACUC). After 14 days of the in vitro culture, the Sam2 gene was introduced into the neuron by using lipofectamine 2000 reagent. Immunostaining and electrophysiological test were performed on neurons after 17 to 18 days of in vitro culture. The neurons were placed in a fixing solution including 2% formaldehyde, 4% sucrose and 1×PBS for 2 minutes, and then pretreated with cold methanol for 10 minutes. After pretreatment, the sample was allowed to react with primary antibodies (rat anti-HA (Roche; 1:250), mouse anti-vGAT (Synaptic systems; 1:250), mouse anti-vGLUT (Millipore; 1:250), rabbit anti-GABAAR 2 (Synaptic systems; 1:500), mouse anti-PSD-95 (NeuroMab; 1:400), mouse anti-gephyrin (Synaptic systems; 1:1,000), mouse anti-Gal (Promega; 1:1,000), alc rabbit anti-Gal (Abcam; 1:5,000)) and secondary antibodies (Alexa Fluor488 (LifeTechnologies), Cy-3 and Cy5-conjugated (Jackson ImmunoResearch Laboratories)) for a day. Then, the electrophysiological test was performed by measuring miniature inhibitory postsynaptic current (mIPSC) through whole-cell patch recordings after Sam2 gene overexpression. In addition, mIPSC was analyzed by using the Mini Analysis software.

Consequently, as shown in FIG. 14, it has been found that, as a result of investigation using inhibitory synapse markers (vesicular GABA transporter (vGAT), gephyrin, and GABA_{A} receptor a2 subunit (GABA_{A}R a2)) after Sam2 gene overexpression, the number and expression level of the inhibitory synapses are significantly decreased than those of the control. In particular, it has been found that the influence of the Sam2 gene on the inhibitory synapse was equally applied to neighbor neurons in addition to the neuron treated with Sam2, and therefore the Sam2 gene is a secretory protein (FIG. 14).

<6-2> Investigation of Excitatory Neuron Marker Through Sam2 Gene Overexpression

To investigate expression of an excitatory neuron marker through Sam2 gene overexpression, immunostaining was performed.

Specifically, to investigate expression of an excitatory neuron marker through Sam2 gene overexpression, excitatory neuron markers, i.e., vesicular glutamate transporter (vGLUT) and PSD-95 were analyzed. Neurons were fixed in a fixing solution including 2% formaldehyde, 4% sucrose and 1×PBS for 2 minutes, and dehydrated for 10 minutes by using methanol stored at −20° C. Then, washing was sequentially performed by using 75%, 50%, and 25% methanol/PBS solution. Primary antibodies (for inhibitory synapse marker: mouse anti-vGAT (Synapse system, 1:250), rabbit anti-GABAARα2 (Synapse system, 1:500), mouse anti-β-Gal (Promega, 1:1,000); for excitatory synapse marker: mouse anti-PSD-95 (NeuroMab, 1:400), mouse anti-vGLUT (Millipore, 1:250) were added to the washed neurons, and reacted. The resultant was washed by using 1×PBS, and then secondary antibodies (Alexa Fluor488 (LifeTechnologies), Cy3- and Cy5-conjugated (Jackson ImmunoResearch Laboratories)) were added and reacted. Thereafter, neurons stained with the inhibitory and excitatory synapse markers were observed under a fluorescent microscope.

Consequently, as shown in FIG. 15, it has been found that the Sam2 gene acts as a neurotransmitter to thereby increase the activity of the nervous system such as decline in presynapse inhibitory function and enhancement of excitatory synapse (FIG. 15).

### Example 7

### Investigation of Autism and Hyperanxiety Symptom in Human Due to Sam2 Gene Loss

<7-1> Recruit of Patients

Copy number variation was investigated by performing array comparative genome hybridization (aCGH) to 35,000 patients.

<7-2> Investigation of Autism and Hyperanxiety Symptom in Human Due to Sam2 Gene Loss Caused by Microdeletion

To investigate autism and the hyperanxiety symptom due to Sam2 gene loss caused by microdeletion in patients recruited through the method as described in Example <7-1>, the following method was conducted.

Specifically, autism and the hyperanxiety symptom due to Sam2 gene loss were diagnosed through counseling with a doctor and patients recruited through the method as described in Example <7-1>.

Consequently, as shown in FIG. 16, it has been found that patients have autism and the hyperanxiety symptom (excessive fear) due to Sam2 gene loss caused by microdeletion are identified in the human and therefore the Sam2 gene is an important factor in the human to control the anxiety response (FIG. 16).

1. A method for preventing or treating a mental disease, regulating the activity or expression of a samdori protein that has at least 90% sequence similarity to the amino acid sequence of samdori2 (sam2) according to SEQ ID NO: 29 (GTCEVIAAHRCCNKNKIEERSQTVKCSCFPGQVAGTTRAAPSCVDASIVAQKWWCQMQPC MDGEECKVLPDLKGWSCSTGNKVKTT).

2. The method for preventing and treating a mental disease of claim 1, wherein the mental disease is anxiety syndrome, depression, autism, manic-depressive illness, schizophrenia, mood disorders, sleep disorders, or attention deficit hyperactivity disorder (ADHD).

3. A method for identifying a cellular receptor protein of a samdori protein, the method comprising:
1) treating a samdori protein that has at least 90% sequence similarity to the amino acid sequence of samdori2 (sam2) according to SEQ ID NO: 29 (GTCEVIAAHRCCNKNKIEERSQTVKCSCFPGQVAGTTRAAPSCVDASIVAQKWWCQMQPC MDGEECKVLPDLKGWSCSTGNKVKTT) with cells;
2) measuring activity or expression of a cellular receptor protein in cells of step 1); and
3) identifying the cellular receptor protein of step 2) as a cellular receptor of the samdori protein when the activity or expression of the receptor protein was increased or decreased.

4. An animal model for a mental disease in which a samdori gene is knocked out in an animal other than a human.

5. The animal model for a mental disease of claim 4, wherein the animal is a mouse, a rat, or a zebrafish.

6. The animal model for a mental disease of claim 4, wherein the mental disease is anxiety syndrome, depression, autism, manic-depressive illness, schizophrenia, mood disorders, sleep disorders, or attention deficit hyperactivity disorder (ADHD).


SEQUENCE LISTING (29 sequences)

SEQ ID NO: 1 - DNA, 1937 bp, Danio rerio
cccgaatgcactggagtggggatggtccatcggcaactataaactgattctcatcaggaaactgcacattatctccccatcacttcaaaggtctcgtcaggcagaggtgacgccaggagatgatttaaaggtgaaaatgacaaggtttccacccctcaaaccttggctccttttctgacaatacagtctgaatgaacccgatgtctttttttttactgtggaaataggatcggaagagagtaacatttttttttttttaatcctgataaagaagattgttgggaagctctttgaaaaaaaattttaaattgtggcacagatggattttaaaaagtgttagatctttccaatgaacactaatagagtactctgctcttggctggatttttcagagaatggcaatggtctctgcgatgtcctgggtcctgtatttgtggataagtgcttgtgcaatgctactctgccatggatcccttcagcacactttccagcagcatcacctgcacagaccagaaggagggacgtgtgaagtgatagcagcacaccgatgttgtaacaagaatcgcattgaggagcggtcacaaacagtaaagtgttcctgtctacctggaaaagtggctggaacaacaagaaaccggccttcttgcgtcgatgcctccatagtgattgggaaatggtggtgtgagatggagccttgcctagaaggagaagaatgtaagacactccctgacaattctggatggatgtgcgcaacaggcaacaaaattaagaccacgagaattcacccaagaacctaacagaagcatttgtggtagtaaaggaaaaccaaccctctggaaaatacattttgagaatctcaaacatctcacatatatacaagccaaatggatttcttacttgcactttgactggctaccagataatcacagtgcgtttactgtgtgtaacgaaatatcctacagtgagaagacacagcgttttggcaacaccatggaaagtgggcttaaaaaagggttttctcagtgaaatttttgggcatcatgaagaacgatcaactatcttctaatttgaatctatagttactttgtaccatttgaaatatatgtatatatatatatataatattttgaaatattatctattctcttcaagaaatgaacagtaccacagtttgagacggctggtgtacccctttgagttttggatgttttgtctgttttgctttgttttgttagtcatttctttttctaacggcaaggaagatatgtgcccttttgagaattcaagatggcactgacacgggaaggccagctacaggtggactcctggaatttgaggcatcataatgatactgaatcaagaacttccttctgcttctaccagatggcccaaggaagcacatcgtcctgttttattgctttctaccctgtgcaatattagcatgcaagcttggcttacatagtcatactttatattcaattgatatataataaccgttctaacctcttccaggaaaatatttttagaactactagcttttccacttagaagaaaatgaggattcttaagggagccactccaccatgctattaagactctggcagagttatgggtaggatatggatccctacatgaataagtcctgtaaatacaatgtcttaaggctttgtatagctgtcctagactgcagaaatgtcctctgattaaatccaaagtctggcatcgttaactacatagtgctgtagcaacaagtcttatcatggcatctctttctatgtttggtttgctttttccaagagtattcaggtctcctcttgtgagataggaaggccatgaaaacaattagatttcaagatgatctatgtgaccaaatgttggacagccctattaaagtggtaaacaacttctttctaaaaaaaaaaaaaaaaaaaaaaaaa

SEQ ID NO: 2 - DNA, 4069 bp, Danio rerio
gcagttgctggggctaacagaactggctgttgggagagagaggcgaggcaacgccgcccggccctgccattccattttactgcttaactcaacctggagtgtcaggacctattctcctgcattctcgctgcactatacgggggagccaagtcactctgtactcactggacagccaggctgggtggaatagggttctagagtggagcaggggctctgccctttatggcgactttctaaaagattagcatcagtgtcttttgacagtatagcatctggggtgggacgcggagaggaggctttgcttgctaaacggcttctttctccgccggtgatctggatttgtttcctcggccccctccccccgctcctttctttctcccctcccgcctttagcaaagtgacacaggcgacacctgctcgcttgtgttcgatgttggaactcgcacctcctcggtggtgacggtgccagggcactgctaccagggggacatcggcgggtttcccttccttccagcccacgatgcctagaagggcagcgggtgggctgcgtggggctctgcccgcagcacttcgaggcgggattgaggggctgaagctggccaggagttgctgctaagtggattcgagtggaaggcgccaagtctccgcgagggcaccagcggggacctatctgcgagcagtgggaacaggggcacctggacggaggaacgcgggcgtctggaggggggacatcggctgagctcagagcctctcctccttcctgtcccgagcttcccagcaactccgccacgttggagacccggtacctgcgagagccagggaactcgagaagtgcgtggccgaggctgctttcctgaaggtgaatcattctgtccaattgcctttccccagagaacaagagggagggcgggagagagggaacggaggatgtgtgagtgtgtgtgtgtgtgagagggagagacgacgtgaggcgagaggaaaagtctttagtcggccttaaaagcaaacaaatcagagatggaataagagcggtaattgcagcaagaccgccttgattcttgcagtccagggagctgagcgcaccgcgcgcatccggcgaggacaggaggcgaccgcgggcgctgccaagggctgcgggactttggcttttcctcagtaaacaaatcttttgattactttgacactgtggaataaagaagcggggagaaggatcaggctcaccttcacccgcttcagggggattccagcttggatgtcagattcctgaaccgtctttgccatcggaggaggaaacacatccacacacgcgcgcgcacactcgcacgctcagggccacactcacacgccgccctccacgatcacacagcaccagacttcggttccctatgcccctggatggtgacggcggattggcatcttggaagcgatgcgagagcgataaggctggcgccggcccgcaaaagctgcaggagcatcgctaggtgttgccgccaccgggaagcggggctgcaggatgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggtaacccattaacccaggagaaatcaagtgatcctcaaggctgatgacattgaacatgcgcatagaaacttaactcaactcctgaggtgatcttgaagatttttataccacttgaaagaggcgctcaatagtctatttccaagggatttcatggcctcttcttgaaatcaagactttttaaaagtcagacatgaacttgcatgtcatgaagatttcagcagatttgaactgtgttcaacttgtaaattgttaaaagaatttgaagtcactgtctgaggagctggtgaagagttgtttttttcagggtgatgttagagacagtcaccttttgagttattggctccagatgtgactacttttcttgtttctgcaagctgtatcccaagtgcactgtccttctgtcctggatgtgttcctgggtcctatgttcatttgctagtgggactacacatggctttaatgacatttcctttgagaacttttcctctggcatggtgtagactgagacaattttatttatatcctaatcttggagctcagaaagcctacatgttttaacatcttaaagttgcttttgttaaaggaatggaaatatatatccattggtaataatgttggcaagtaatagttatctgaataaatcaatcatataagaatgtatagacaagctgacatatttccctaaggctaacaacaccctgctgaagctctttgtcaaataggtagtagttagaactggattgccattttcattatataatactttgtacctctagagcactctccctttctgtttttttttaagtgagcttttctttaattttttatgtttacttattcccttcacagaaatcagcagtgagcagtcaagttaatgggtagccttcagtttcaaaaaaattgacagggatgcatgtgagtttctgatttcttagcttgaacattattcacttagatttcttccagtattttttaaaaaactgtcctatctcattttaaaagactttcttttgcttgatcccaatgactgtttgaatgcttatatatttgttcaatctgttgatagaaaaaattgttcattttcctcagtctcaaatttataaatatttgcttacagttttcctattcaaacaatttgttaggccaatattttgtgacatttttgtagcgattttaacgtttatggttttggttctacaggaaagtcataaatatttaaaggccttaaacatgtatgtactttttttttctaagttatagaatgtataattttgtactacatttattttgtttcatttgtgatatgaagggagagaagaaagaaaagtgcatagccattctgtaacaatattgtgtaaacctatagtttgaaggaatgcaaggagaaggatttctgtgttttactcattttaggctgttcagaagatgcttcaaaaattgtcctgttagaatttccatcatggaaggtggtatggaagaaggtatggaaatactttgtattctaaaaactcactgacgtggtcagttagacatacgttggtttccaggatagaggcccatatatcctggggagctttggtctattagtttgtgacaatattcaaaggccaaaacactactcagacactttcctgggaagagcaactaaaaatgtaaaattggttaaaaataaaatctgaaaagtatgtatctcacattgaactaaaatccactgtctcataagttcatggaatgaaatggctttctgcctccattttaatcatgcataaaatgaattagatggctttgagtggattttcacaatggctcaagactatatgaaattataaaaaaaaagttgccctggggtttctgcatcaattagaatatcattaatttctttgtaaccaagtgaaaaactatactttttggaaattatgaatttgtcctaggtttgtttgagatttgaaattatacatcatgcttctcattttttaaactatgttctttaaatcaacactggaaactctgtattatatacaagtgtaatacatgcatataatagaaaaaaaacatggaatttcaaatatactaactagattatccccagtagattaatgttgtgactattcagaaaaggtgaataaaattgggatataaaatggactctctttcataaaaaaaaaaaaaaaa

SEQ ID NO: 3 - DNA, 1077 bp, Danio rerio
acttgtccaggtggagtgagtctgaggacagcagatgaacagacagaaactgaaagatccccaaaaaggatgagtgagagggtcgagcggaactggagcacgggcggctggctgctggcactgtgcctggcctggctgtggacccacctgaccttggctgccttgcagcctcccactgccacagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacgcctccatcgtcctgcagagatggtggtgtcagatggagccctgcctgccgggggaggagtgtaaggtgctcccggacctgtcgggatggagctgcagcagtggacacaaagtcaaaaccaccaaggtcacacgatagctcttgggggtcacggcctggacaagaaaggcttgactgagccgtgaactgaagaatggtatccagtcatcagccaggaaagatggggattcacttacatgcctcatgtcaaatgcagcatcagtctttccggggcatttcagttaagctgctcagcagatatggatggatctgcaatcacatacctaatgtggagctgggcttttctggagacacgaaggtcaacacacaattcctgcccttaaggaatgtccagttgaattggagagttgatgacagacaatttagataatttaggttaaagtacttgataccagactgcggcttctgggccacatgctatggcatgatgggggtttgggaatgagattcccacagttcttcagataccctgtggccacagggcatagaaacaagaggtcacattcagcacccaccacctccctctttcgcatcagttctgaatccccagcaagctgttaacatgttgcaggaaaacactctccccttatgccagaccagcagtatctcatttggatgggattggattgacttgcggaaggaaagtaaaaataaagccaaataacttcccaaaaaaaaaaaaaa

SEQ ID NO: 4 - protein, 133 aa, Danio rerio [sequence omitted]

SEQ ID NO: 5 - protein, 131 aa, Danio rerio
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRVTH

SEQ ID NO: 6 - protein, 133 aa, Danio rerio
MSERVERNWSTGGWLLALCLAWLWTHLTLAALQPPTATVLVQQGTCEVIAAHRCCNRNRIEERSQTVKCSCFSGQVAGTTRAKPSCVDASIVLQRWWCQMEPCLPGEECKVLPDLSGWSCSSGHKVKTTKVTR

SEQ ID NO: 7 - DNA, 26 bp, Artificial
  Sam2_Forward
acgccgctgaatgaaccgattaccgg

SEQ ID NO: 8 - DNA, 24 bp, Artificial
  Sam2_reverse
gagcgaacgcactgctttacacac

SEQ ID NO: 9 - DNA, 23 bp, Artificial
  Beta-actin_forward
gaggagcaccccgtcctgctcac

SEQ ID NO: 10 - DNA, 23 bp, Artificial
  Beta-actin_reverse
gatggctggaacagggcctctgg

SEQ ID NO: 11 - DNA, 20 bp, Artificial
  Sam2cnu1_Forward
tctactgaggagtggtgtga

SEQ ID NO: 12 - DNA, 20 bp, Artificial
  Sam2cnu1_Reverse
ggtcagtttcagagagctgg

SEQ ID NO: 13 - DNA, 12 bp, Artificial
  Left ZFN
tgctcctgcttc

SEQ ID NO: 14 - DNA, 12 bp, Artificial
  Right ZFN
caggtggcagga

SEQ ID NO: 15 - DNA, 2248 bp, Danio rerio
tgggctcggctccgcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtagcgaagagagaggtgtgcttcaatcctggaggggcagcaggaggcggagctcttttgcttggattcccatcatggcccctctgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttatagcctaacagttatttgatgttattcttgcagagggaatggaaaggaatggaaagatttgttggcgtaatttttgaatatttgttatgatcatatgaataagtaaaaaaattcatcctgctgatggcaaaaaaaaaaaaaaaaa

SEQ ID NO: 16 - DNA, 2615 bp, Danio rerio
gcggcggcggcggaggatggcgcgcgcggggcccgcacgtggaggccggcgcgggggcgcgggcagggccggctgctgagacgcgctgctgccccccgcgcgggcgccgcggcttcaatggcgccatcgcccaggaccggcagccggcaagatgcgaccgccctgcccagcatgtcctcaactttctgggcgttcatgatcctggccagcctgctcatcgcctactgcagtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctgacaaacacagcccctgaggggccccgggagtggccttggctccctggagagcccacgtctcagccacagttctccactcgcctcggacttcacccgttctctgccgcccgcccactccgtttccctgtggtccgtgaaggacggcctcaggccttggcatcctgagcttcggtctgtccagccgacccgaggaggccggactcagacacataggcggggggcggcacctggcatcagcaatacgcagtctgtgggagcccggccgcgcccagcccccgccgaccgtggcgttggccctgctgtcctcagaggaggaggaggaggaggcagctccggcagccacagaaggctgcagcccagcccgcctgagacacgacgcctgccccaggggactgtcaggcacagaagcggcctcctcccgtgccccagactgtccgaattgcttttattttcttatactttcagtatactccatagaccaaagagcaaaatctatctgaacctggacgcaccctcactgtcagggtccctggggtcgcttgtgcgggcgggagggcaatggtggcagagacatgctggtggccccggcggagcggagagggcggccgtggtggaggcctccaccccaggagcaccccgcgcaccctcggaggacgggcttcggctgcgcggaggccgtggcacacctgcgggaggcagcgacggcccccacgcagacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagcaaagaaaactcgagtaacacttgtttgaaagagatcattaaatgtattttgcaaagcctaaagttatatatttaacagtttttatatgttgtatatttgtagaaaatcctatttaacaattaacgtggcagtcccggccgtcctgagagtcgggccgagccccgtgtgtttctgaagactctgggggtgggacacggcggggaggtggtgccccgcggaccccggggtgccaggcacggaaggcgggactctgggagaagcgtgcggaggaccgtggcgtcggcgtcccggatgtgtcggtcgtgcccggggaggccgggttcccctcgctgcgggccaggcttggctcctgattccctctctggtccctgtattggtcaacacttgagcgtacaatatcttgaacatgcttcttccaatgggttttgtttcccatttcctgcccctttcgccactcacggaccttgaggccagttgacggcccttctccccacgcctgtgtccccgcgttctgagaagtcctctgtcttcgtgtcactaggtccagaaagtcgcgccgggcagaggcgcaggcggggccggcagggccgaggaataagcgacaattctggtttttctcccctggccgtcgttcgccagcctccttcattttcctgagttcccgctgaagtatatactacctatgagtccaattaacatgagtattatgctagttctatcctactaaaaaaaacgtaaaaaaataactatatagaagctgttccagcaaccatagactgaagatacgaaagaaaatccatttatttaagacctgttccggtatccatgaggacataatttacctttcagtcaccacaaatttataggcatttgtatcctggactaaaagaaggggctgaggttgggtttgtcatcacagagggggtgggcctggaaagggtccttcccaagctgccccggctccggcggcccgggccggcagcctctgccagccagcgtcctcacggcctccccctcgcctgtttcttttgaaagcaagtgtagacaccttcgagggcagagatcgggagatttaagatgttacagcatatttttttttcttgttttacagtattcaattttgtgttgattcagctaaattatgaaaaataaagaaaaactcctttgataaaaaaaaaaaaaaaa

SEQ ID NO: 17 - DNA, 2652 bp, Danio rerio
aaagacacaaatcgcctcccggagtggcgcctccagtcgcggcggagcgcggcgttggcggcggatggagggcgcgagcgggcggccgcggaggctgcacccggcggggcgctgatgcggcgcctggaccttcgctgcgcgacttcgggggcgtcggccgagttgggactccgcgatgcagctcctgaaggcgctctgggcactggcaggggccgcgctctgctgcttcctcgtcctagtgatccacgcgcagttcctcaaagaaggtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctgacaaacacagcccctgaggggccccgggagtggccttggctccctggagagcccacgtctcagccacagttctccactcgcctcggacttcacccgttctctgccgcccgcccactccgtttccctgtggtccgtgaaggacggcctcaggccttggcatcctgagcttcggtctgtccagccgacccgaggaggccggactcagacacataggcggggggcggcacctggcatcagcaatacgcagtctgtgggagcccggccgcgcccagcccccgccgaccgtggcgttggccctgctgtcctcagaggaggaggaggaggaggcagctccggcagccacagaaggctgcagcccagcccgcctgagacacgacgcctgccccaggggactgtcaggcacagaagcggcctcctcccgtgccccagactgtccgaattgcttttattttcttatactttcagtatactccatagaccaaagagcaaaatctatctgaacctggacgcaccctcactgtcagggtccctggggtcgcttgtgcgggcgggagggcaatggtggcagagacatgctggtggccccggcggagcggagagggcggccgtggtggaggcctccaccccaggagcaccccgcgcaccctcggaggacgggcttcggctgcgcggaggccgtggcacacctgcgggaggcagcgacggcccccacgcagacgccgggaacgcaggccgctttattcctctgtacttagatcaacttgaccgtactaaaatccctttctgttttaaccagttaaacatgcctcttctacagctccatttttgatagttggataatccagtatctgccaagagcatgttgggtctcccgtgactgctgcctcatcgataccccatttagctccagaaagcaaagaaaactcgagtaacacttgtttgaaagagatcattaaatgtattttgcaaagcctaaagttatatatttaacagtttttatatgttgtatatttgtagaaaatcctatttaacaattaacgtggcagtcccggccgtcctgagagtcgggccgagccccgtgtgtttctgaagactctgggggtgggacacggcggggaggtggtgccccgcggaccccggggtgccaggcacggaaggcgggactctgggagaagcgtgcggaggaccgtggcgtcggcgtcccggatgtgtcggtcgtgcccggggaggccgggttcccctcgctgcgggccaggcttggctcctgattccctctctggtccctgtattggtcaacacttgagcgtacaatatcttgaacatgcttcttccaatgggttttgtttcccatttcctgcccctttcgccactcacggaccttgaggccagttgacggcccttctccccacgcctgtgtccccgcgttctgagaagtcctctgtcttcgtgtcactaggtccagaaagtcgcgccgggcagaggcgcaggcggggccggcagggccgaggaataagcgacaattctggtttttctcccctggccgtcgttcgccagcctccttcattttcctgagttcccgctgaagtatatactacctatgagtccaattaacatgagtattatgctagttctatcctactaaaaaaaacgtaaaaaaataactatatagaagctgttccagcaaccatagactgaagatacgaaagaaaatccatttatttaagacctgttccggtatccatgaggacataatttacctttcagtcaccacaaatttataggcatttgtatcctggactaaaagaaggggctgaggttgggtttgtcatcacagagggggtgggcctggaaagggtccttcccaagctgccccggctccggcggcccgggccggcagcctctgccagccagcgtcctcacggcctccccctcgcctgtttcttttgaaagcaagtgtagacaccttcgagggcagagatcgggagatttaagatgttacagcatatttttttttcttgttttacagtattcaattttgtgttgattcagctaaattatgaaaaataaagaaaaactcctttgataaaaaaaaaaaaaaaa

SEQ ID NO: 18 - protein, 140 aa, Danio rerio
MRSPRMRVCAKSVLLSHWLFLAYVLMVCCKLMSASSQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 19 - protein, 132 aa, Danio rerio
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 20 - protein, 125 aa, Danio rerio
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 21 - DNA, 436 bp, Danio rerio
cagccggtgctcctgccatgtcctggctcctgtgtctgtggataagtgttagctgcttactgctctgtcacgccactttatacgaaaccatccagcagcatcatgtgacccgtccgggccgaaacgccatccagatactggaaggaggaacatgtgaggtgatcgcagcacaccgatgttgcaacaagaaccgtattgaggaacgttcccagacggtcaagtgttcatgtttaccaggaaaagtggctggcacgacacgaaacaaaccctcctgtgtagacgcctccattgtgattgggaagtggtggtgtgagatggagccatgtctggagggagaagagtgcaagacgctaccagacaactccggctggatgtgttcctctgggaataagatcaagaccacgaggattcatccccggacctaacaaagagctcc

SEQ ID NO: 22 - DNA, 883 bp, Danio rerio
gggcgattcgatatcgcggccgcgatccgacgaaagtgagaagacagaagaactgagagtttctacaagacagcaatacctggacgaaaggatatccggacacctcacaatgaatggattgaggacaaagtgttgagttttacatacagctgtccctggacgatacagaggagtgttgggatgtcgtggctgctgtgggtttgggtcagtctgacagtcctgctgatgtgtgagggcactttctatgagaccatccagcagcacctggcaccgcctggaaccaacatacagatcctcgaaggagggacatgcgaggtgatcgcggcgcacaggtgctgtaacaaaaacaagattgaagagagatctcagacagtgaagtgctcctgtttacctgggaaagtggcaggaaccaccaggaacaaaccctcctgtgttgacgcctccattgtgattgggaagtggtggtgtgagatggagccgtgtctggagggagaagagtgtaagactctaccagacaactccggctggatgtgctactctggaaacaagatcaaaaccaccaggaatacccacccatacacatccttgtagcagaggtagaatcactgcaaagctcaaaatggaggaactttgaatgaagggaaacatccaagaggacaagctgaaataagttttcccaactctgacccaacaaccatgtgtctaaagacttgaaatggaatcctcggctcataaatccatctgtctctgagtaacacggacaacagactctttaagaaagtcgttgtgggcgtgaacactgctggctcttcttccctctgtttttgtttgacagctgtcaatattgtttcctctcagtggataattaaactcgtatcccttt

SEQ ID NO: 23 - DNA, 505 bp, Danio rerio
acgccgctgaatgaaccgattaccggatcgatgaacccccgagtcccgctggactgatcgggctgatcggatggattgatcaggagttaatcagagctgcaggatgagtgtccgagatcttcttctgcttctctctcagactcttcttctgcttctgcttctcctgagatcttcagctcactccacagagcttcacattgttgaggtgcggacgggcacctgtgaggtcgtgtctttacatcgctgctgcaacaagaacaagatcgaggagcgttcgcagaccgtcaagtgctcctgcttcccgggacaggtggcaggaacaaccagagccgccccgtcctgcgtcgacgctgctatcgtccagcagaagtggtggtgtcagatgcagccgtgtctggatggtgaagagtgtaaagttctgccggatctgaaaggatggagctgcgccaccggaaacaagatcaagaccaccaagatctctccagaccgagatcagctgtggtga

SEQ ID NO: 24 - DNA, 1580 bp, Danio rerio
cgcttcaagtgctgaacacttgaggggcgagtcgagctcctcaacgccgctctcagtctccacgcagcctgaacacaagactacagcagccaaaaacaccactggtccatcatggataaggggatattgccgatttgaaaccttcgttataatacccgcaattaagggtctcgtctttacgtgcttattcagatccgttccggcgcgcaaagttcagacgcatctgaaggaactttggagagctggagttcagaggactcaatcgagacttttctgaactgaagcaggaatcgctttaataccatttgcattctcgtttgtttgagtctgtaggtgatgcttttcctgggatcgtgtttgcgcgaagggaaattatcaggctttgccactgccggggttttacttctgacaatgaagagcaaatgagagcagtattacttcacctttattgacagctcaacgacaggatgcgggagagggagctgcagcgtgtgggcaggagattgctgctgttgggcatctgtctgctgtctctctggggtcagttcagcctggcctcgacacatcgcagccatattgtgcacgtcaaagcgggaacctgtgaggtcattgctgcccaccgctgctgtaacaagaacaaaatcgaggagcgctcccaaactgtcaagtgctcctgcttccctgggcaagtggctggaacaacaagagctgctccatcctgtgtggacgcatctatagtagcgcagaagtggtggtgtcagatgcagccgtgtatggacggtgaggagtgtaaggtgttgccggatctaaaaggctggagctgcagcacaggcaacaaagtcaagacgaccaaggtcacacgatagtctgggaccaacatacatttgggactagcagcattttttttctctctccccattttttcgatcaaagcaatggaaatgaagatacatagacttcaaactacttcacaactatcgggatcaacttgttggacctcaactgaatactaatgtcacttggaatagaccatttaaacacatggagtgttttctctctcttccccttgtcctcttctacagtattttcactctgccagactcctgtcttttgttttataaaagtgtcggacaagatcatacgccatgtaaagtaaatattacatttaaaaaaaatcagttcagagacaattctgccaccatggattataacggatttcgtttttcaagcgaaagtggactcgagaggatcacttattacttaaatcatatgttccagaggcactaatattcattccactatccttcagtgccaatattttctttcttctactatgagcagaatttttgatcagtgtgaggtgtatgtttttttggctatggttttgaaacgggacttgtgcaaaaacaaatcgcaagtgttatgataagaattacaggtattaaacctgataaaatgtgtacgggagctgtaaaaaagatggcaaaaaagatttattgtttgtttttaaaagccttggtggccaatattgagtcaaagtgtcagtattatctcaaataaaactctatctctatggaaaa

SEQ ID NO: 25 - DNA, 1728 bp, Danio rerio
caggtttagacgagtcttaaggctactcaggtggccctgtgcatttagatggcagaattaatgcaagtcaggtgaacctgtaaaatattcagaaacaacgtgagctcagaaagagaagaagctgcaggacagctgtcacaaacgccgctgagtggcgctgattcaccgctcattgcgtttcttcagaacatccctggagagcacagggatcgagtcagcgcagcgcagctccagacctttgccttcagcaccggcagatcctgcacagagcgcgaacgcgtctccagctcggacaaacactaggaaaacaagcccaaggggatactacgcgagatgagggctccgttacaaacatctgtgtgatttttaaaccaggagcgaaatcgttgatgtgaagtttcacgcagctctccgttgagttgagatgaagatctgagccgctgtgagagaaggaactttgcgtcttcgcgcacccggacgccctgcggggacatcgggcgcttcagtgataagaggactgaacttcttttctgtatttggggctttctagcttactctgacagagtagacacagaagggatcctttggtgcggattgttgaagcatctccagttcgtggatggaataaatcataatggaagggcaatgagacctctttctgctgctgggcaataatccggactaccgcgccaacaggatgcaggagcgggcgctgcagagggcgtttgcgtggatcgtactggtatttttggcccttgtgttgttttggggtcatttgtctgaagcgtcttctcaccgcagtcacatcgtgcacgtgaaagctggcacatgtgaggtgattgcagcccatcgctgctgtaacagaaataagattgaggagcgatcgcagacggtcaagtgttcctgcttccccggacaagtggctggaacgacgagagctgcaccctcctgtgtggacgcatctatagtagctcagaagtggtggtgtcaaatgcagccatgtgtggatggtgaggagtgtaaagtcctgcccgacctcacaggctggagctgcagcacgggcaacaaggtgaagacaaccaaggtgactcgatagttccaagcaacaatgtcctgggacaaaagagctgaaaatggagactaactagaggcagcattgagcttcagctaacaggatttatcagtattagtcatatgaagattatctgcaatcaggaacagactgatctcctccataaacattaggaccatcctagagttaatgtgcccttctcaatggaccccctggccttccggacgctcgatctttcgaatgtggatcaatagacgctggaatgtgttggctagggggtcttgatgttcatatgcagtttatttgttttttacagaaggtttggatcacctacaacagtgcagcccagtggagtttaagctcttcagtaacatgctctccgtcattgtatttcagttctccccacaattccatgattatccctgctctacctgaccataaggtccaattattccttgtggaatcagcatttaaacgtctggacctctaatggactcatgtgtatagtctttatttctaaatgaaaagcttttcagccccacagtctgaaacaacagattagatgtacaagtatattctagataatgtaaaagtgtaaaagatcaaagtgttattctcaaaaacaagcgaaataaagtttaacggttatcactttt

SEQ ID NO: 26 - DNA, 863 bp, Danio rerio
aaaagaaagtaagctcgtttaaaacaaacatctgtcttcaggactcctgctacatcccgaagcgaaggagaatgctttcgctttcataaacggaggatctgagcggtgtttgaggacacaggaggagagacatgtccagctcacagagaagagactgatcagacaacacagaaagatgcagctgtggggtcgatctctgtggccgctgcggattgtgctcttgttcaccttggtaatgtgttcgtgtgggttggtttctgcgggcacgcggagtctgcggggacacacagcttcataccaggtaaagcagggtacctgtgaggtggttgccatccaccgctgctgcaataagaacaagattgaggagcgttcacagaccgtcaagtgctcatgtttccctggtcaggtggccggtaccacacgtgcacagccatcttgtgtggaggcttccatcgtgctacagaagtggtggtgccaaatgcacccatgtttggatggcgaggagtgcaaagctcttccagatctgactggctggtcctgcagcaccggtaacaaagtcaaaaccactaaggtcacacgatagcaacaacatggcagttctaccatgaggcccaagactccttaacgacccactggaaaagaaaactttcttgtttaagacactgacgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtg

SEQ ID NO: 27 - DNA, 1673 bp, Danio rerio
acagacggaggcggagatacagacacggcgtttctccaagaacacacgcgtctcgtttgggaccttttacacgaccgttcgaaataatgcgggaattacggctatttcgaagctgactttatctgaatgtaggctttacagcaagcgctgtcgacaggctactgtacagaaaacagttcacagatcgcgcagttctcccagtgcgtaaaacagacggacaggatggtcgcgcatcacttcacttagatccacgcaacagctacagacaaactggaaatttcagctcctttttttctccgatggaagctctaaaatataaaatatgttattttcctaaagtttctattttttgaaagaaaacggattgctttctttctgaatcttcaaacgaaaacgtagcagaggtgttggttgttgtatggcattagaatggagatgtggcgtggatgctgaaggcagtcaggatgctgatgctgagagttgcgtgggctctagcgggagcggcggtctgctgttttctcatcgttctcatccactctcgcttcctaagagacggtcaacttgcagcaggcacctgtgagattgtcactttagacaaagacagcagtcagccacgcaggacgattgccagacagacagctcgctgtgcatgtaagaaaggacagattgctgggacaacgaatgccaggccggcctgtgtggacgctcgtatcgtgaagaccaagcagtggtgtgacatggtgccctgtctagaggatgaagagtgtgacttgttagtaaataaatctggctggacttgtacacagccaagtggcagagtgaaaactacaacggtgtcctaacagtgggaggggttcctggagcgctaccagacaaggcctcttgcagatttgcccacctgctgtagatacttgctatgtctcatcggcttgacacctgcctctgcctaccagtctgcgtctcagctttgtaaatgggcagtacccccttgtgccacggagagcatactagatgttcacaaagcgcctcatggacagcagcagccttgaggaatttatgagcgactctgaggcacctaccctgtttgagactgaaccaaacataggcttttttgggactccaccagcggttctgacctagactgtgaaccaagccgctttttgaactgctttctgctgaagtacatttttgccactcatttttacagaccaaagaggaaaaaataatgatataatacagaaatttaaaaaaatccattgactatgttagcacggactgagagacagacttgaaacgattaagacggtgaaaaaaatatatatatacctaaatgtagagaggtagatatagatctatgaagactttgctatacagcggcggtctacaagcaaaaatgctgacatggaacactgaaacgactgggttgaccattcccatttatcacaatctgatgagaacctttaatattttacagatttccttgaaaggaactacccactgcatttcaaggacagctaggatatagatactgtacacaatcacatcaacaaatttgtttagcatttttcagtagcaacattttggacagtttgcattcagcatggaaggttaaatggaaccgtaccccattgtttatcgtcagaagatattaaacaagaacatgaaaactgtca

SEQ ID NO: 28 - DNA, 408 bp, Danio rerio
acgcgtcagatcctcggaaagatgcagcatctccgtgtggcgtgggcgctcgcgggagcggcggtgggcttttttctgctcttcttgatccaagcgcatttcttcggcgaaggtcagctggcggcaggtacatgtgaaatcgtgactctggaccgggacagcagccagccaagaaggaccatcgcccggcagacggccaggtgcgcctgcaggaagggccagattgcgggcactaccagagccagtccagcatgtgtggatgcgcaaattgtcaggaccagacagtggtgtgagatgatgccttgtttggatgacgaaggctgtgacctattggtaaacagaacaggctggacgtgtgcacagccaggaggccgagtgaaaacaaccacggtatcctgacgccagtgg

SEQ ID NO: 29 - protein, 86 aa, Danio rerio
GTCEVIAAHRCCNKNKIEERSQTVKCSCFPGQVAGTTRAAPSCVDASIVAQKWWCQMQPCMDGEECKVLPDLKGWSCSTGNKVKTT